(19) United States
(12) Patent Application Publication
(10) Pub. No.: US 2006/0078531 A1
(43) Pub. Date: Apr. 13, 2006

(54) Method of prevention and treatment of atherosclerosis, peripheral vascular disease, coronary artery disease, and age-related disorders including osteoporosis, arthritis, type 2 diabetes, dementia and Alzheimer's disease

(76) Inventor: Osemwota Sota, Tarzana, CA (US)

Correspondence Address: Osemwota Sota Omoigui, 4019 W. Rosecrans Ave., Hawthorne, CA, 90250, US

(21) Appl. No.: 10/961,037

(22) Filed: Oct. 12, 2004

(51) Int. Cl. A61K 31/785; A61K 31/675; A61K 31/66; A61K 31/455; A61K 31/366; A61K 31/401; A61K 31/192

(52) U.S. Cl. 424/78.14; 514/89; 514/102; 514/59; 514/355; 514/423; 514/460; 514/548; 514/571

(57) ABSTRACT

This invention relates to a method for prevention and treatment of Atherosclerosis, Peripheral Vascular Disease, Coronary Artery Disease, and age-related disorders including Osteoporosis, Arthritis, Type II Diabetes, Dementia and Alzheimer's disease in a subject comprising administering to said subject a therapeutically effective dosage of each component or combination of statins, bisphosphonates and/or cholesterol lowering agents or techniques, administered separately, in sequence or simultaneously. Cholesterol Metabolites (isoprenoids) are an integral component of the signaling pathway for Interleukin 6 mediated inflammation. Interleukin 6 inflammation is the common causative origin for Atherosclerosis, Peripheral Vascular Disease, Coronary Artery Disease, and age-related disorders including Osteoporosis, Arthritis, Type II Diabetes, Dementia and Alzheimer's disease. Said method for prevention and treatment of said disorders is based on inhibition of Interleukin-6 inflammation through regulation of cholesterol metabolism and isoprenoid depletion.

### BACKGROUND OF THE INVENTION

This invention relates to a method of prevention and treatment of atherosclerosis, peripheral vascular disease, coronary artery disease, and age-related disorders including osteoporosis, arthritis, type 2 diabetes, dementia and Alzheimer's disease, by inhibition of the cholesterol pathway. Cholesterol Metabolites (isoprenoids) are an integral component of the signaling pathway for Interleukin 6 mediated inflammation. Interleukin 6 mediated inflammation is the common causative origin for Atherosclerosis, Peripheral Vascular Disease, Coronary Artery Disease, and age-related disorders including Osteoporosis, Arthritis, Type 2 Diabetes, Dementia and Alzheimer's disease.

### DESCRIPTION OF THE PRIOR ART

The current theories and treatment options for Atherosclerosis, Peripheral Vascular Disease, Coronary Artery Disease, and age-related disorders including Osteoporosis, Arthritis, Type 2 Diabetes, Dementia and Alzheimer's disease are fragmented and not satisfactory. There is currently no unifying theory that links Cholesterol metabolites and Interleukin-6 inflammation as the common causative origin for all the above diseases. As such current strategies for each disease entails different medications and therapeutic procedures such as statins, aspirin, beta blockers, ACE inhibitors and angioplasty for atherosclerosis and coronary heart disease, statins and thrombolytics for peripheral vascular disease, oral hypoglycemics for Type 2 diabetes, bisphosphonates and calcitonin for osteoporosis, and Acetylcholinesterase inhibitors e.g. rivastigmine, donepezil and galanthamine for dementia and Alzheimer's disease.

### SUMMARY OF THE INVENTION

The present invention provides a method for the prevention and treatment of Atherosclerosis, Peripheral Vascular Disease, Coronary Artery Disease, and age-related disorders including Osteoporosis, Arthritis, Type 2 Diabetes, Dementia and Alzheimer's disease, in a human or other animal subject. Therapeutic targets for the prevention and control of all of the above diseases should focus on Cholesterol metabolites and Interleukin-6 mediated inflammation. Prevention and treatment of the above diseases will be by utilization of cholesterol lowering agents or techniques and/or treatment with Statins and/or Bisphosphonates to inhibit Interleukin-6 inflammation through regulation of cholesterol metabolism.

### DESCRIPTION OF THE DRAWINGS

FIG. 1. Isoprenoid Synthesis

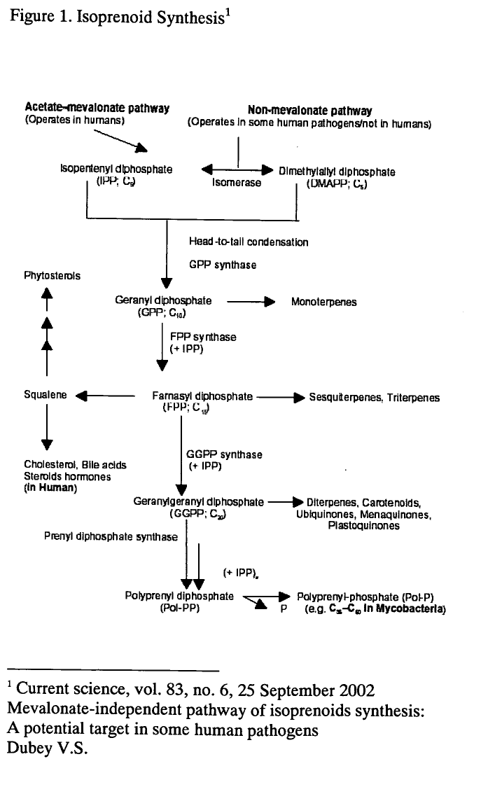

### DETAILED DESCRIPTION OF THE INVENTION

Cardiovascular disease (CVD) is the leading cause of death and disability in developed nations and is increasing rapidly in the developing world. By the year 2020, it is estimated that CVD will surpass infectious diseases as the world's leading cause of death and disability. Atherosclerotic vascular disease (ASVD), which encompasses coronary heart disease, cerebrovascular disease, and peripheral arterial disease, is responsible for the majority of cases of CVD in both developing and developed countries . One of the most important advances in medicine has been the identification of the major risk factors for CVD, which arose from large prospective cohort studies such as the Framingham Heart Study and the Seven Countries Study. The major modifiable risk factors include elevated blood pressure, dyslipidemia, smoking, and diabetes mellitus. A substantial body of evidence now supports reducing these factors to reduce morbidity and mortality associated with ASVD. Indeed, screening for and treating these conditions forms the basis of many published guidelines of risk assessment and reduction strategies.

Despite changes in lifestyle and progress in pharmacological cholesterol lowering, cardiovascular disease remains the leading cause of death and illness in industrialized countries and will become the most prevalent health problem worldwide³. Atherosclerosis, a progressive disease characterized by the accumulation of lipids and fibrous elements in the arteries, constitutes the single most important contributor to this growing burden of cardiovascular disease. The link between lipid metabolism and atherosclerosis dominated the thinking until the 1980s⁴. Over the last fifteen years, however, a prominent role for inflammation in the pathogenesis of atherosclerosis has been established⁵. Now atherosclerosis is considered as an inflammation-mediated disease driven by complex interactions between leukocytes, platelets and cells of the vessel wall. Endothelial injury is the first and crucial step in the pathogenesis of atherosclerosis. A plethora of genetically determined and epigenetic factors, such as oxidized low-density lipoprotein (LDL), free radicals (e.g., due to cigarette smoking), hypertension, diabetes mellitus, elevated plasma homocysteine, infectious microorganisms, autoimmune reactions, and combinations thereof, have been identified as etiological principles. Endothelial injury triggers inflammation with increased adhesiveness and activation of leukocytes (mainly monocytes) and platelets, which is accompanied by the production of cytokines, chemokines, vasoactive molecules and growth factors.

The lesions of atherosclerosis occur principally in large and medium-sized elastic and muscular arteries and can lead to ischemia of the heart, brain, or extremities, resulting in infarction. The earliest type of lesion, the “*fatty streak”, which is common in infants and young children, is an inflammatory lesion, consisting of monocyte-derived macrophages and T-lymphocytes. In persons with hypercholesterolemia, the influx of these cells is preceded by the extracellular deposition of amorphous and membranous lipids.

The cells of most organs and tissues satisfy their requirements for membrane cholesterol via endogenous cholesterol biosynthesis^{6 7}. Many cell types, however, have acquired mechanisms to internalize exogenous sources of cholesterol, usually in the form of plasma-derived lipoproteins⁸ Examples include steroid-synthesizing cells, hepatocytes, and macrophages and smooth muscle cells in atherosclerotic lesions, often referred to as foam cells. A portion of the cholesterol in such cells is either synthesized de novo, internalized as lipoprotein (a)⁹ or taken up as VIDL or LDL via the LDL receptor. However, most of this cholesterol probably originates from cell detritus ingested during phagocytosis and from chemically modified lipoproteins taken up via the scavenger receptor and the putative receptor for OxLDL^{10 11}. To be recognized by the scavenger or OxLDL receptors, LDL must undergo chemical modification, a process that probably occurs in the microenvironment of the subendothelial space¹². Such chemically modified IDL acts as an irritant. For example, many of the compounds found in OxLDL are highly reactive and have cytotoxic effects. The formation of foam cells is also associated with the production of numerous immune mediators that further intensify the inflammatory process¹³ In the case of steroidogenic cells, the internalization of lipoprotein-cholesterol represents a physiological process that provides cells with precursor cholesterol stores, to be used for “acute” steroid hormone production¹⁴. Hepatocyte lipoprotein uptake mediates the clearance of various classes of plasma lipoproteins ¹⁵, which can lead to whole-body elimination of excess diet derived cholesterol in the bile, a process known as reverse cholesterol transport 16. The uptake of arterial-wall lipoproteins by macrophages and smooth muscle cells may be a type of physiological scavenging response that initially helps rid the endothelium of potentially harmful lipoprotein material¹⁷. This cellular process eventually contributes to the progression and complications of atherosclerotic vascular disease. Cells that rely totally or mostly on endogenous cholesterol synthesis cannot accumulate excess endogenous cholesterol because of homeostatic regulation at multiple steps in the cholesterol biosynthetic pathway¹⁸. Cells that internalize exogenous cholesterol also repress endogenous cholesterol biosynthesis and LDL receptor expression in response to cholesterol loading. Furthermore, these cells have evolved other mechanisms to prevent the accumulation of excess unesterified, or “free,” cholesterol (FC). One mechanism is cholesterol esterification, which is mediated by the microsomal enzyme acyl-coenzyme A: cholesterol acyltransferase (ACAT)¹⁹. The two forms, ACAT-1 and ACAT-2, differ in their sites of expression, with macrophages and most other cell types expressing ACAT-1. In humans, intestinal epithelial cells, but not hepatocytes, selectively express ACAT-2; mice, in contrast, express ACAT-2 in both of these cell types²⁰. Another important protective mechanism against FC accumulation is cellular efflux of cholesterol and certain cholesterol-derived oxysterols^{21 22}. In addition, some of the physiological pathways described above, such as steroid and bile acid biosynthesis, may help limit the accumulation of intracellular FC in steroidogenic cells and hepatocytes, respectively²³ The evolution of multiple pathways to prevent intracellular FC accumulation reflects the toxic effects of excess cellular FC²⁴. The hallmark of the early atherosclerotic lesion is the Cholesterol ester-laden (CE-laden) macrophage foam cell²⁵. Progressive FC loading of lesional macrophages leads to a series of phospholipid related adaptive responses. These adaptive responses eventually fail, leading to macrophage death. Macrophage death by either necrosis or apoptosis leads to lesional necrosis, release of cellular proteases, inflammatory cytokines, and prothrombotic molecules, which could contribute to plaque instability, plaque rupture, and acute thrombotic vascular occlusion²⁶. Indeed, necrotic areas of advanced atherosclerotic lesions are known to be associated with death of macrophages, and ruptured plaques from human lesions have been shown to be enriched in apoptotic macrophages.

The presence of apoptotic and necrotic macrophages in atherosclerotic lesions has been well documented in many human and animal studies^{27 28}

What is missing in the current theories and thinking is the direct molecular link (exclusive of macrophage death), between cholesterol and inflammation, and between Inflammation and Atherosclerosis, Peripheral Vascular Disease, Coronary Artery Disease, and age-related disorders including Osteoporosis, Arthritis, Type 2 Diabetes, Dementia and Alzheimer's disease.

The acute phase response occurs prior to antibody-mediated immunological defense. It occurs in response to an inflammatory response brought on by injury and trauma, neoplasm, or disordered immunological activity. A local reaction at the site of injury or infection leads to an activation of cytokines (specifically, IL-6, IL-1, TNF-[Alpha], and interferons) that triggers a systemic response consisting of leukocytosis; increases in glucocorticoid production; increases in erythrocyte sedimentation rates, fever, activation of complement and clotting cascades; decreases in serum zinc and iron; and an increase in plasma levels of acute phase proteins, C-reactive protein (CRP), serum amyloid A, fibrinogen, and other proteins²⁹ CRP is one of the two most abundant acute phase reactants in humans: levels rapidly increase in the circulation as a result of either trauma or infection. Manufactured in the liver and deposited in damaged tissue, CRP is found in high levels in inflammatory fluids and in both the intimal layer of the atherosclerotic aortic artery and the foam cells within the lesions of atherosclerotic plaque.³⁰ CRP has been found in the fatty streak of the aortic artery and is key in many of the inflammatory sequences that promote the progression of atherosclerosis^{31 32} CRP stimulates mononuclear cells to release tissue factor, an identified protein that is central to the initiation of coagulation reactions, complement activation, and the neutralizing of platelet-activation factor. Together, these reactions promote a thrombotic response.³³

Levels of cytokines involved in the acute phase response—TNF-Alpha, IL-1, IL-6, and fibrinogen—have been shown to be elevated in cases of unstable angina related to aneurysm^{34 35 36} and have been positively correlated with the risk of primary and recurrent myocardial infarction and death^{37 38 39}. The risk associated with these elevated levels remains constant even when the data is adjusted for other major risk factors: blood pressure, total and HDL cholesterol, body mass index, diabetes, alcohol use, family history, and exercise frequency⁴⁰. Elevated levels of highly sensitive C-reactive protein (hs-CRP) have been related to increased risk of cardiovascular disease, myocardial infarction, and coronary artery disease (CAD) deaths among individuals with angina pectoris^{41 42 43}. Assayed levels of hs-CRP can increase 100 times over normal levels within 24-48 hours after an acute inflammatory stimulus. However, in long term prospective studies inter-individual variations in hs-CRP levels may occur over long periods of time, in the absence of trauma or acute infection⁴⁴ Elevated levels of hs-CRP have shown a doubling of risk both for ischemic stroke in hypertensive men and women^{45 46} and for peripheral artery disease⁴⁷ Some of the most convincing data on hs-CRP has come from the Women's Health Study, a prevention trial using both vitamin E and aspirin⁴⁸ Of 12 markers studied, hs-CRP was the most sensitive predictor of a cardiovascular event: death from heart disease, myocardial infarction, or surgical revascularization. Women who were in the highest quartile of hs-CRP were 4.4 times more likely to experience an event than women in the lowest quartile. Even in women with low cholesterol (LDL below 130 mg), hs-CRP still had a predictive effect: women were 4.1 times more likely to experience an event if they were in the highest quartile of hs-CRP levels. When all markers were simultaneously controlled for, the only markers able to independently predict coronary events were hs-CRP and total cholesterol:HDL ratio. The combined predictive effects of these two measurements were greater than either one alone and could predict women who were six times more likely to experience a future cardiovascular event. This predictive effect held true in women who were matched for smoking status, age, body mass index, hypertension, diabetes, and parental history. In this study smoking did not effect the predictive value of hs-CRP.

Recent studies are now demonstrating that IL-6 and TNF-alpha are stronger predictors of cardiovascular disease than C-reactive protein. In the Health, Aging and Body Composition study⁴⁹, done at the Wake Forest University School of Medicine, the researchers tracked the medical history of the 2,225 participants for an average of 42 months after measuring their blood levels of C-reactive protein, IL-6 and TNF- alpha. People with the highest IL-6 levels were two to five times more likely to have a heart attack, stroke or other cardiovascular episode than those with the lowest levels. High blood levels of TNF-alpha increased the risk of heart disease by 79 percent and of heart failure by 121 percent. High levels of C-reactive protein increased the risk of heart failure by 160 percent compared to those with low levels, but they did not significantly raise the risk of a first stroke or heart attack.

As expected, the incidence of cardiovascular disease was high for people with the conventional risk factors—smoking, high blood pressure, high cholesterol and the like. But for participants free of those risk factors, the inflammation-related molecules were better predictors of heart disease. In another study of 3,045 well-functioning persons aged 70 to 79 years, participating in the Health, Aging and Body Composition study⁵⁰, the same researchers investigated the association of several inflammatory markers with subclinical and clinical cardiovascular disease in older men and women. IL-6 levels were associated with the highest risks for subclinical cardiovascular disease as well as for clinical cardiovascular disease

Cholesterol Metabolism

Normal healthy adults synthesize cholesterol at a rate of approximately 1 g/day and consume approximately 0.3 g/day. A relatively constant level of cholesterol in the body (150-200 mg/dL) is maintained primarily by controlling the level of de novo synthesis. The level of cholesterol synthesis is regulated in part by the dietary intake of cholesterol. Cholesterol from both diet and synthesis is utilized in the formation of membranes and in the synthesis of the steroid hormones and bile acids. The greatest proportion of cholesterol is used in bile acid synthesis⁵¹. Cholesterol synthesis occurs in the cytoplasm and microsomes from the two-carbon acetate group of acetyl-CoA. The process has ten major steps:

1. Synthesis begins when acetyl-CoA is derived from an oxidation reaction in the mitochondria and is transported to the cytoplasm

2. Two moles of acetyl-CoA are condensed, forming acetoacetyl-CoA. Acetoacetyl-CoA and a third mole of acetyl-CoA are converted to 3-hydroxy-3-methylglutaryl-CoA (HMG-CoA) by the action of HMG-CoA synthase.

3. HMG-CoA is converted to mevalonate, in a rate limiting step catalyzed by the enzyme HMG-CoA reductase, (HMGR)

4. Mevalonate is then activated by three successive phosphorylations, yielding 5-pyrophosphomevalonate

5. After phosphorylation, an ATP-dependent decarboxylation yields isopentenyl pyrophosphate, (IPP), an activated isoprenoid molecule. Isopentenyl pyrophosphate is in equilibrium with its isomer, dimethylallyl pyrophosphate, DMAPP.

6. One molecule of IPP condenses with one molecule of DMAPP to generate geranyl pyrophosphate, (GPP)

7. GPP further condenses with another IPP molecule to yield farnesyl pyrophosphate, (FPP). This step is catalyzed by FPP synthase

8. FPP condenses with another IPP molecule to yield geranylgeranyl pyrophosphate (GGPP). This step is catalyzed by GGPP synthase

9. The head-to-tail condensation of two molecules of FPP yielding Squalene, is catalyzed by squalene synthase.

10. Squalene undergoes a two-step cyclization to yield lanosterol.

11. Lanosterol is converted to cholesterol, through a series of 19 additional reactions.

There is a complex regulatory system to co-ordinate the biosynthesis of cholesterol with the availability of dietary cholesterol. The cellular supply of cholesterol is maintained at a steady level by the following mechanisms:

1. Regulation of HMGR activity and levels

2. Regulation of excess intracellular free cholesterol through the activity of acyl-CoA:cholesterol acyltransferase, (ACAT)

3. Regulation of plasma cholesterol levels via LDL receptor-mediated uptake and HDL-mediated reverse transport.

Regulation of HMG-CoA reductase (HMGR) activity is the primary means for controlling the level of cholesterol biosynthesis. Transcriptional regulation of cellular cholesterol biosynthesis is via a protein called the Sterol Regulatory Element Binding Protein (SREBP) that exists as an integral membrane protein in the Endoplasmic Reticulum. When the level of free cholesterol is low, a protease cleaves off a portion of the protein that is in the cytoplasm and the cleaved portion migrates to the nucleus where it binds to DNA sequences that regulate the expression of genes involved in cholesterol and lipid synthesis. One of the genes regulated in this manner is HMG-COA reductase.

Cholesterol is transported in the plasma predominantly as cholesteryl esters associated with lipoproteins. Lipoprotein complexes contain a core of highly apolar triglycerides and cholesterol esters surrounded by a monolayer of phospholipids. The proteins of the lipoproteins are embedded in the particle. The phospholipid is a monolayer surrounding a droplet of triglyceride and cholesterol ester with the hydrophobic tails of the lipids inserted into the droplet. Cholesterol ester contains a fatty acid esterified to the hydroxyl group of the cholesterol. There are a variety of proteins, termed apolipoproteins, in lipoprotein particles. At least one important function of some of these proteins is to recognize receptors on the surface of cells; for example, apolipoprotein B-100, a protein of molecular weight 500,000, is prominent in low density lipoproteins (LDLs) and is responsible for the binding of LDL to receptors on the surface of cells that take up the LDL. There are several different types of complexes distinguished on the basis of their density: a) High density lipoproteins (HDL). b) Low density lipoproteins (LDL). c) Intermediate density lipoproteins (IDL). d) Very low density lipoproteins (VLDL). e) Chylomicrons.

Dietary cholesterol is transported from the small intestine to the liver within chylomicrons. Cholesterol synthesized by the liver, as well as any dietary cholesterol in the liver that exceeds hepatic needs, is transported in the serum within low density lipoproteins (LDLs). The liver synthesizes very low density lipoproteins (VLDLs) and these are converted to LDLs through the action of endothelial cell-associated lipoprotein lipase. Cholesterol is delivered to cells by the LDL lipoprotein particle. Apolipoprotein B-100 in LDL binds to a specific receptor on the surface of cells, the LDL receptor. After binding, LDL is taken into the cell within a vesicle by the process of receptor-mediated endocytosis. After endocytosis, LDL is delivered to lysosomes where there is hydrolysis of the fatty acid of the cholesterol ester by the enzyme acid lipase to produce free cholesterol.

Cholesterol found in plasma membranes can be extracted by high density lipoproteins (HDLs) and esterified by the HDL-associated enzyme Iecithin: cholesterol acyltransferase (LCAT). The cholesterol acquired from peripheral tissues by HDLs can then be transferred to VLDls and LDLs via the action of cholesteryl ester transfer protein (apo-D) which is associated with HDLs. Reverse cholesterol transport allows peripheral cholesterol to be returned to the liver in HDLs. Ultimately, cholesterol is excreted in the bile as free cholesterol or as bile salts following conversion to bile acids in the liver⁵².

Activation of Interleukin-6 Inflammation by Cholesterol Metabolites

Isoprenoid precursors are necessary for posttranslational lipid modification (prenylation) and, hence, the function of Ras and other small guanosine triphosphatases (GTPhases)⁵³. These GTPase proteins such as Ras, Rho, Rac, and Rab (particularly Rho) are intracellular signaling proteins that, when activated, are involved in receptor-coupled transduction of signals from extracellular stimuli to cytoplasm and the nucleus. The Rho proteins belong to the Ras superfamily. The Ras proteins alternate between an inactivated GDP-bound form and activated GTP-bound form, allowing them to act as molecular switches for growth and differentiation signals. Prenylation is a process involving the binding of hydrophobic isoprenoid groups consisting of famesyl or geranylgeranyl residues to the C-terminal region of Ras protein superfamily.

Farnesyl pyrophosphate and geranylgeranyl pyrophosphate are metabolic products of mevalonate that are able to supply prenyl groups. The prenylation is conducted by prenyl transferases. The hydrophobic prenyl groups are necessary to anchor the Ras superfamily proteins to intracellular membranes so that they can be translocated to the plasma membrane⁵⁴. The final cell-membrane fixation is necessary for Ras proteins to participate in their specific interactions⁵⁵. The activity of the small GTPase, Rac1, plays a role in various cellular processes including cytoskeletal rearrangement, gene transcription, and malignant transformation. The IL-6 receptor system consists of an IL-6 specific binding molecule, IL-6R and a signal transducer, gp130. Following gp130 dimerization, IL-6 activates multiple signaling pathways (Ras dependent MAP Kinase cascade, STAT1-STAT3 heterodimer pathway, and STAT3 homodimer pathway). Several other cytokines belonging to the Interleukin-6 family including oncostatin M, IL-11, leukemia inhibitory factor (LIF), ciliary neurotrophic factor (CNTF) and cardiotropin-1 (CT-1) use gp130 as a common signal transducing molecule and therefore have similar biological activities^{56 57 58} In one study⁵⁹, constitutively active Rac1 (Rac V12) is shown to stimulate the activation of STAT3, a member of the family of signal transducers and activators of transcription (STATs). The activity of Rac1 leads to STAT3 translocation to the nucleus coincident with STAT3-dependent gene expression. The expression of Vav (Delta1-187), a constitutively active guanine nucleotide exchange factor for the Rho GTPases, or activated forms of Ras or Rho family members, leads to STAT3-specific activation. The activation of STAT3 requires tyrosine phosphorylation at residue 705, but is not dependent on phosphorylation of Ser-727. The study indicated that Rac1 induces STAT3 activation through an indirect mechanism that involves the autocrine production and action of IL-6, a known mediator of STAT3 response. Rac V12 expression results in the induction of the IL-6 and IL-6 receptor genes and neutralizing antibodies directed against the IL-6 receptor block Rac1-induced STAT3 activation. Furthermore, inhibition of the nuclear factor-kappaB activation or disruption of IL-6-mediated signaling through the expression of IkappaBalpha S32AS36A and suppressor of cytokine signaling 3, respectively, blocks Rac1-induced STAT3 activation. These findings elucidate a mechanism dependent on the induction of an autocrine IL-6 activation loop through which Rac1 mediates STAT3 activation establishing a link between oncogenic GTPase activity and Janus kinase/STAT signaling.

In another study⁶⁰, leukemic cells from 50 patients with acute myeloid leukemia (AML) were analyzed for the presence of activating point mutations of the N-RAS gene using polymerase chain reaction (PCR) and differential oligonucleotide hybridization. This assay allows semiquantitative determination of the relative abundance of cells carrying N-RAS mutations. Clonal activation of N-RAS, noted in the large majority of leukemic cells of the six of these patients, was correlated significantly (p=0.0003) with the ability of these cells to express interleukin 6 (IL-6), previously shown to be expressed at high levels in approximately 30% of primary AML cells.

Activation of Interleukin-6 Inflammation by Activated Monocytes in the Inflammatory Response to Infection and Trauma

HMG-CoA reductase generates mevalonate, the precursor of a complex series of isoprenoids molecules that are necessary for posttranslational lipid modification (prenylation) and, hence, the function of intracellular signaling proteins that, when activated, are involved in expression of Interleukin 6 mediated inflammation. Tissue injury, subsequent to a physical, chemical or biological insult, results in an inflammatory response associated with invasion of the area by immune cells, which include monocytes, T helper cells, lymphocytes, neutrophils, eosinophils, and other cells such as fibroblasts and endothelial cells. Isoprenoids are required for NADPH oxidase activity in granulocytes via LMW GTP-binding protein isoprenylation. Isoprenoid generation through the mevalonate pathway is a requirement for IL-8 and IL-6 induction by activated monocytic cells in vitro. One study evaluated the effects of isoprenoid depletion on the expression of proinflammatory genes in human monocytic THP-1 cells. The researchers selected conditions under which pretreatment for 24 h with isoprenoid synthesis inhibitors (HMG-CoA reductase inhibitor lovastatin or compactin at 10 microM) did not compromise cell viability but markedly suppressed H2O2 generation. Under these conditions interleukin-8 (IL-8) production was attenuated (by 50-90%) in response to lipopolysaccharide, granulocyte-macrophage colony-stimulating factor, and phorbol myristate acetate. Coincubation of reductase inhibitor-treated cells with mevalonate prevented the attenuation of IL-8 production by reductase inhibitors. The effects of isoprenoid depletion on cytokine production were selective: IL-11 beta generation was not inhibited but the production of IL-6 and IL-8 was concomitantly suppressed. IL-8 induction was suppressed at least in part through attenuation of the increase in mRNA in stimulated cells. The study authors concluded that isoprenylation inhibitors have the potential to alter monocyte proinflammatory function⁶¹. In another study, fluvastatin decreased (and mevalonate rescued) signaling molecules within membrane rafts in monocytes in parallel with effects on tyrosine phosphorylation events. In addition, Fcgamma receptor mediated immune complex trafficking, activation of MAP kinases (ERK and p38), and downstream inflammatory mediator release (MMP-1 and IL-6) were blocked by fluvastatin. The study authors concluded that HMG-CoA reductase inhibition alters immune receptor signaling in monocytes by disrupting membrane rafts essential for the initiation of signal transduction⁶². Another study explored the role of mevalonate inhibitors in the activation of NF-kbeta and the induction of inducible nitric oxide synthase (iNOS) and cytokines (TNF-alpha, IL-1beta, and IL-6) in rat primary astrocytes, microglia, and macrophages. Lovastatin and sodium phenylacetate (NaPA) were found to inhibit Lipopolysaccharide (LPS) and cytokine-mediated production of NO and expression of INOS in rat primary astrocytes; this inhibition was not due to depletion of end products of mevalonate pathway (e.g., cholesterol and ubiquinone). The authors stated that reversal of the inhibitory effect of lovastatin on LPS-induced iNOS expression by mevalonate and farnesyl pyrophosphate and reversal of the inhibitory effect of NaPA on Lipopolysaccharide (LPS)-induced iNOS expression by farnesyl pyrophosphate suggests a role of farnesylation in the LPS-mediated induction of iNOS. The inhibition of LPS-mediated induction of INOS by FPT inhibitor II, an inhibitor of Ras farnesyl protein transferase, suggests that farnesylation of p21(ras) or other proteins regulates the induction of iNOS. Inhibition of LPS-mediated activation of NF-kbeta by lovastatin, NaPA, and FPT inhibitor II in astrocytes indicates that the observed inhibition of iNOS expression is mediated via inhibition of NF-kbeta activation. In addition to iNOS, lovastatin and NaPA also inhibited LPS-induced expression of TNF-alpha, IL-lbeta, and IL-6 in rat primary astrocytes, microglia, and macrophages. The authors concluded that their study delineates a novel role of the mevalonate pathway in controlling the expression of iNOS and different cytokines in rat astrocytes, microglia, and macrophages that may be important in developing therapeutics against cytokine- and NO-mediated neurodegenerative diseases⁶³.

Bacterial infection as typified by periodontal disease is associated with inflammation and the inflammatory response, with generation of isoprenoids by activated monocytes. Bacteria also directly synthesize isoprenoid molecules by a mevalonate—independent (non-MVA) pathway. The synthesis of IPP and DMAPP via the non-MVA pathway starts with the formation of 1-deoxy-Dxylulose-5-phosphate (DOXP) by two glycolytic intermediates, pyruvate and glyceraldehyde-3-phosphate⁶⁴. These isoprenoids may be involved in the cell-wall biosynthesis and may also play a role in direct activation of biologically active mediators⁶⁵. Periodontal disease is characterized by adherence and colonization of the tooth enamel and root surface by saccharolytic, aerobic Streptococcus species and other bacteria. This sets the stage for Fusobacterium nucleatum to coaggregate with these early colonizers and to permit late colonizers, including dental pathogens, to eventually form a biofilm. These complex interactions result in the release of factors that lead to tooth decay⁶⁶. In a landmark study in Finland, Matilla et al examined the role of chronic bacterial infections as risk factors for coronary heart disease. The association between poor dental health and acute myocardial infarction was investigated in two separate case-control studies of a total of 100 patients with acute myocardial infarction and 102 controls selected from the community at random. Dental health was graded by using two indexes, one of which was assessed blind. Based on these indexes dental health was significantly worse in patients with acute myocardial infarction than in controls. The association remained valid after adjustment for age, social class, smoking, serum lipid concentrations, and the presence of diabetes⁶⁷. More recently, these results were confirmed in studies in the United States, Canada, Great Britain, Sweden, and Germany. In another study, Morrison et al found that people with periodontal disease had a factor of 2 higher risk of dying from cardiovascular disease. By comparison smokers only had a 60% increased risk⁶⁸. Meyer et al showed that c-reactive proteins and pro-inflammatory cytokines are released during periodontal flare-ups and capable of eliciting effects associated with atherosclerosis and coronary heart disease The presence of oral infections is also associated with cerebrovascular disease, stroke⁷⁰, preterm births⁷¹, osteoporosis⁷² and type 2 diabetes. One study evaluated 113 Pima Indians with both diabetes and periodontal disease. The study found that when their periodontal infections were treated, the management of their diabetes markedly improved⁷³.

Inhibition of Cholesterol Pathway by Statins

The main effect of statins is the decrease of serum level of low-density lipoprotein (LDL) cholesterol, due to the inhibition of intracellular cholesterol biosynthesis. A minor effect is the decrease of serum triglycerides. Statins decrease the production of mevalonate, geranyl pyrophosphate, and farnesyl pyrophosphate, and subsequent products on the way to construction of the cholesterol molecule. Thus, statins could inhibit inflammation, by inhibition of the cholesterol pathway and intracellularly interfering with Ras superfamily protein function⁷⁴. Ikeda et al.⁷⁵ recently showed that statins decrease matrix metalloproteinase-1 expression through inhibition of Rho. Statin therapy has been demonstrated to provide significant reductions in non-high-density lipoprotein cholesterol, and to decrease cardiovascular morbidity and mortality.

Inhibition of Cholesterol Pathway by Bisphosphonates

Recent findings suggest that alendronate and other N-containing bisphosphonates inhibit the isoprenoid biosynthesis pathway and interfere with protein prenylation, as a result of reduced geranylgeranyl diphosphate levels. One study⁷⁶ identified farnesyl disphosphate (FPP) synthase as the mevalonate pathway enzyme inhibited by bisphosphonates. High-performance liquid chromatography (HPLC) analysis of products from a liver cytosolic extract narrowed the potential targets for alendronate inhibition (inhibitory concentration at 50% [IC(50)]=1700 nM) to isopentenyl diphosphate isomerase and farnesyl diphosphate synthase. Recombinant human farnesyl diphosphate synthase was inhibited by alendronate with an IC(50) of 460 nM (following 15 min preincubation). Alendronate did not inhibit isopentenyl diphosphate isomerase or GGPP synthase, partially purified from liver cytosol. Recombinant farnesyl diphosphate synthase was also inhibited by pamidronate (IC(50)=500 nM) and risedronate (IC(50)=3.9 nM), negligibly by etidronate (IC50=80 microM), and not at all by clodronate. In osteoclasts, alendronate inhibited the incorporation of [(3)H]mevalonolactone into proteins of 18-25 kDa and into nonsaponifiable lipids, including sterols. The authors concluded that the findings (i) identify farnesyl diphosphate synthase as the selective target of alendronate in the mevalonate pathway, (ii) show that this enzyme is inhibited by other N-containing bisphosphonates, such as risedronate, but not by clodronate, supporting a different mechanism of action for different bisphosphonates, and (iii) document in purified osteoclasts alendronate inhibition of prenylation and sterol biosynthesis. In another study, a wide range of bisphosphonates, were found to have a significant correlation between potency for inhibition of recombinant human FPP synthase in vitro and anti-resorptive potency in vivo, suggesting that this enzyme is the major pharmacologic target of these drugs. The most potent anti-resorptive bisphosphonates such as zoledronic acid and risedronate are very potent inhibitors of FPP synthase, with IC50 values as low as 3 nM and 10 nM respectively. Inhibition of FPP synthase prevents the formation of FPP and its derivative GGPP. These isoprenoid lipids are necessary for the post-translational lipid modification (prenylation) of small GTPase proteins such as Ras, Rho, Rac, and Rab. The effects of nitrogen-containing bisphosphonates on osteoclasts can be overcome by addition of components of the mevalonate pathway, which bypass the inhibition of FPP synthase and restore protein prenylation. In particular, geranylgeraniol (a cell-permeable form of GGPP), prevents inhibition of resorption by nitrogen-containing bisphosphonates in vitro.⁷⁷ . Another study demonstrated that the major molecular target of nitrogen-containing bisphosphonate drugs is farnesyl diphosphate synthase, an enzyme in the mevalonate pathway. In an in vitro screen, the authors discovered a bisphosphonate, NE21650, that potently inhibited farnesyl diphosphate synthase but, unlike other N-BPs investigated, was also a weak inhibitor of isopentenyl diphosphate isomerase. NE21650 was a more potent inhibitor of protein prenylation in osteoclasts and macrophages, and a more potent inhibitor of bone resorption in vitro, than alendronate, despite very similar IC(50) values for inhibition of farnesyl diphosphate synthase. The study observations show that minor changes to the structure of bisphosphonates allow inhibition of more than one enzyme in the mevalonate pathway and suggest that loss of protein prenylation due to inhibition of more than one enzyme in the mevalonate pathway may lead to an increase in antiresorptive potency compared to bisphosphonates that only inhibit farnesyl diphosphate synthase.⁷⁸ One study aimed to evaluate cholesterol and lipoprotein serum levels in patients with Paget's bone disease treated with intravenous pamidronate. The study included 20 consecutive patients (mean age, 67.6±11.0 years) with Paget's bone disease for at least 1 year, who needed intravenous amino bisphosphonate treatment; 12 patients with inactive Paget's bone disease served as controls. The patients with active Paget's bone disease underwent three cycles (every 3 months) of treatment with 60 mg of intravenous pamidronate. Controls were given a saline infusion following the same administration schedule. In all subjects total alkaline phosphatase (total ALP), bone alkaline phosphatase (bone ALP), total cholesterol (TC), tryglycerides (TG), and high- and low-density lipoprotein cholesterol (HDL-C and LDL-C, respectively) were measured before infusions (pamidronate or saline) at baseline and at 3-month intervals up to 9 months. In the control group no significant changes were observed through the study period for any of the biochemical parameters. In the pamidronate-treated patients, both bone ALP and total ALP significantly fell at the end of the study. In patients with active treatment, at the end of the study period HDLC significantly (P<0.05) increased by 10.3%, whereas IDLC significantly (P<0.05) decreased by 5.5%. In these patients TC showed a negative trend without reaching statistical significance, whereas the HDL-C/IDL-C ratio rose 16.2% above the basal value and TC/HDL-C decreased by 12.5%. The researchers concluded that, pamidronate given intravenously seems to be able to induce a prolonged shifting in circulating cholesterol from the LDL-C to the HDL-C that is associated with a weak decrease in total cholesterol, thus producing a possible improvement in the atherosclerotic risk index

Statins and Interleukin 6

The ability of HMG-CoA reductase inhibitors to lower C-reactive protein levels has recently brought into question the mechanisms of action of the statin drugs. Because these medications lower incidences of acute cardiovascular events as well as decreasing morbidity and mortality well before the effects of lowered LDL cholesterol can be expected to occur, questions have been asked about whether they may work independently of IDL-lowering mechanisms. One study examined the effects of atorvastatin on soluble adhesion molecules, interleukin-6 (IL-6) and brachial artery endothelial-dependent flow mediated dilatation (FMD) in patients with familial (FH) and non-familial hypercholesterolemia (NFH)⁸⁰. A total of 74 patients (27 FH and 47 NFH) were recruited. Fasting lipid profiles, soluble intercellular adhesion molecule-1 (sICAM-1), soluble vascular-cellular adhesion molecule-1 (sVCAM-1), E-selectin, IL-6 and FMD were)measured at baseline, 2 weeks, 3 and 9 months post-atorvastatin treatment (FH--80 mg/day, NFH--10 mg/day). In both groups, compared to baseline, sICAM-1 levels were significantly reduced at 2 weeks, further reduced at 3 months and maintained at 9 months (P<0.0001). The IL-6 levels were significantly reduced at 3 months and 9 months compared to baseline for FH (P<0.005) and NFH (P<0.0001). In both groups, the FMD at 2 weeks was higher than baseline (P<0.005), with progressive improvement up to 9 months. FMD was negatively correlated with sICAM-1 and IL-6. The authors concluded that both low and high doses of atorvastatin lead to early progressive improvement in endothelial function in patients with primary hypercholesterolemia and that sICAM-1 and IL-6 levels reflect endothelial dysfunction in these patients.

Bisphosphonates and Interleukin 6

Because of various modes of action observed in studies, bisphosphonates have been classified into two groups. Bisphosphonates (such as clodronate and etidronate) that closely resemble pyrophosphate—a normal byproduct of human metabolism - are incorporated into adenosine triphosphate (ATP) analogues, which create compounds that are believed to build up and lead to osteoclast death⁸¹. The newest generation of bisphosphonates, which contain nitrogen (such as pamidronate, alendronate, risedronate, and ibandronate), are believed to inhibit protein prenylation (post-translational modification) within the mevalonate pathway. The mevalonate pathway is responsible for the biosynthesis of cholesterol, other sterols, and isoprenoid lipids. Isoprenoid lipids are key in the prenylation of intracellular signaling proteins (GTPases) that, when activated, regulate a number of processes, including osteoclast activity. It is believed that by impeding the function of these regulatory proteins, bisphosphonates block osteoclast functioning and cause apoptosis⁸²

In patients with Paget's disease of bone, bisphosphonate therapy is associated with a significant reduction of Interleukin-6 soluble receptor (sIL 6R) serum levels Bisphosphonates inhibit the production of pro-inflammatory cytokine interleukin-6 in tumoral cell lines of human osteoblastic phenotype (MG63 and SaOs cells), and in peripheral blood mononuclear cells (PBMC)⁸⁴. Bisphosphonates also inhibit IL-1 and TNF-alpha stimulated IL-6 release in cultures of human osteoblastic osteosarcoma cells⁸⁵. Osteoblasts exposed to small amounts of bisphosphonate elaborate a soluble inhibitor, which interferes with osteoclast formation and development⁸⁶. Bisphosphonates prevent apoptosis of murine osteocytic MLO-Y4 cells, whether it is induced by etoposide, TNF-alpha, or glucocorticoid dexamethasone⁸⁷. Pamidronate and other bisphosphonates inhibit the production by osteoblasts of the inflammatory cytokine interleukin-6, a growth factor essential to myeloma cells⁸⁸.

Atherosclerosis and Statins

Changes in intima-media thickness (IMT) and arterial lumen diameter-as measured by B-mode high-resolution ultrasonography and quantitative coronary angiography, respectively-are currently the only surrogate markers for progression of atherosclerotic disease recognized by regulatory authorities in the United States and Europe. Because atherosclerosis is a disease of the arterial wall, the ability of B-mode ultrasonography to provide visualization of IMT offers significant advantages over angiography. These advantages, as well as the safety and noninvasiveness of B-mode ultrasonography, have led to increasing use of this imaging technique in observational studies and interventional studies of lipid-lowering agents over the last decade. These observational studies clearly demonstrated an association between carotid IMT and atherosclerotic disease. Of the interventional studies, the recent Arterial Biology for the Investigation of the Treatment Effects of Reducing Cholesterol (ARBITER) trial found that use of atorvastatin 80 mg daily for aggressive lowering of plasma low-density lipoprotein cholesterol (LDLC) concentrations to below current target levels was associated with significant IMT regression compared with results obtained with less aggressive plasma LDL-C lowering.^{89 90}

Atherosclerosis and Bisphosphonates

Measurement of carotid arterial intima-media thickness (IMT) using B-mode ultrasonography is a noninvasive and powerful tool to evaluate early atherosclerotic lesions^{91 92 93 94 95}. In one study the effect of etidronate treatment on carotid arterial intima-media thickness was prospectively examined in 57 subjects with type 2 diabetes associated with osteopenia. After 1 yr of therapy with cyclical etidronate (200 mg/day for 2 weeks every 3 months), intima-media thickness showed a decrease (mean±SE, −0.038±0.011 mm), which was significantly different from a change in 57 control subjects (0.023±0.015 mm; P<0.005). Cardiovascular parameters were not changed after etidronate treatment. The authors concluded that etidronate in clinical dosage may have an antiatherogenic action, at least in type 2 diabetes, although its mechanisms remain to be elucidated⁹⁶. In another study, administration of ethane-1-hydroxy-1,1-diphosphonate (EHDP) to swine with pre-established atherosclerosis resulted in lower lesion calcium concentration, smaller lesions and a decrease in the area of lesions involved in necrosis. Atherosclerosis was developed in Yorkshire swine by balloon catheter-injury to the abdominal aorta, followed by a high cholesterol-high lipid (HL) diet for 4 months. The administration of EHDP (20 mg/kg/day) was begun after these 4 months and continued for 5 additional months along with the atherogenic diet. Other swine were ballooned and fed HL diet for nine months. Morphometric analysis showed that the extent of lesions, expressed as ratio of intima to media was significantly less (P less than 0.05) in the EHDP-treated HL swine, compared to the HL diet-only group. The ratio of lesion areas showing lipid-rich necrotic debris to the area of media was also significantly smaller (P less than 0.05). Biochemical analysis showed that the lesion from the HL drug-treated group contained significantly less (P less than 0.05) calcium compared to that from the HL diet only. Finally, there was significant correlation between average lesion area and average lesion calcium concentration (P less than 0.02) for both groups. While the effect of EHDP on lesion size and calcium concentration has been previously reported for various species such as rabbit and monkey, the authors concluded that this study is believed to be the first where a beneficial effect of EHDP on one of the most serious complications of atherogenesis—necrosis—has been documented⁹⁷.

Type 2 Diabetes and Interleukin 6

Circulating levels of interleukin-6 (IL-6) are raised in insulin resistant states such as obesity, impaired glucose tolerance (IGT), and type 2 diabetes mellitus (DM). Growing evidence suggests that IL-6 is not only produced by fat cells but is also capable of inducing insulin resistance in these cells. The expected result of this in vivo, would be to increase adipose mass and subsequently body mass index (BMI). The IL6-174G>C common functional gene variant has consistently been associated with increased plasma IL-6, insulin resistance, and increased cardiovascular risk.⁹⁸. In another study, the authors determined whether elevated levels of the inflammatory markers interleukin 6 (IL6) and C-reactive protein (CRP) are associated with development of type 2 DM in healthy middle-aged women. The Women's Health Study, is an ongoing US primary prevention, randomized clinical trial initiated in 1992. From a nationwide cohort of 27 628 women free of diagnosed DM, cardiovascular disease, and cancer at baseline, 188 women who developed diagnosed DM over a 4-year follow-up period were defined as cases and matched by age and fasting status with 362 disease-free controls. Incidence of confirmed clinically diagnosed type 2 DM by baseline levels of IL-6 and CRP. Study results showed that baseline levels of IL-6 (P<0.001) and CRP (P<0.001) were significantly higher among cases than among controls. The relative risks of future DM for women in the highest vs lowest quartile of these inflammatory markers were 7.5 for IL-6 (95% confidence interval [CI],3.7-15.4) and 15.7 for CRP (95% CI, 6.5-37.9). Positive associations persisted after adjustment for body mass index, family history of diabetes, smoking, exercise, use of alcohol, and hormone replacement therapy; multivariate relative risks for the highest vs lowest quartiles were 2.3 for IL-6 (95% CI, 0.9-5.6; P for trend=0.07) and 4.2 for CRP (95% CI, 1.5-12.0; P for trend=0.001). Similar results were observed in analyses limited to women with a baseline hemoglobin A(1c) of 6.0% or less and after adjustment for fasting insulin level. The authors concluded that elevated levels of CRP and IL-6 predict the development of type 2 DM, and the data support a possible role for inflammation in diabetogenesis.

Type 2 Diabetes and Bisphosphonates

Advanced glycation end products (AGE), senescent macroprotein derivatives form at an accelerated rate in diabetes and induce angiogenesis through overgeneration of autocrine vascular endothelial growth factor (VEGF). In this study, effects of incadronate disodium, a nitrogen-containing bisphosphonate on AGE-elicited angiogenesis in vitro, were studied. Incadronate disodium was found to completely inhibit AGE-induced increase in DNA synthesis as well as tube formation of human microvascular endothelial cells (EC). Furthermore, incadronate disodium significantly prevented transcriptional activation of nuclear factor-kappab and activator protein-1 and the subsequent up-regulation of VEGF mRNA levels in AGE-exposed EC. Farnesyl pyrophosphate, but not geranylgeranyl pyrophosphate, was found to completely restore the anti-angiogenic effects of incadronate disodium on EC. These results suggest that incadronate disodium could block the AGE-signaling pathway in microvascular EC through inhibition of protein farnesylation. The authors concluded that Incadronate disodium may be a promising remedy for treatment of patients with proliferative diabetic retinopathy⁹⁹. Charcot neuroarthropathy has been recognized for over 130 years and yet it remains a major cause of morbidity for patients with diabetes mellitus and a continuing challenge for physicians. The underlying cause is thought to be trauma in a neuropathic foot that leads to a complex series of pathological processes culminating in bone and joint destruction and subsequent deformity. A study was undertaken to study the effect of pamidronate, a bisphosphonate, in the management of acute diabetic Charcot neuroarthropathy. Altogether 39 diabetic patients with active Charcot neuroarthropathy from four centers in England were randomized in a double-blind placebo-controlled trial. Patients received a single infusion of 90 mg of pamidronate or placebo (saline). Foot temperatures, symptoms and markers of bone turnover (bone specific alkaline phosphatase and deoxypyridinoline crosslinks) were measured over the 12 months, in 10 visits. All patients also had standard treatment of the Charcot foot. Mean age of the study group (59% Type 2 (non-insulin-dependent) diabetes mellitus) was 56.3±10.2 years. The mean temperature difference between active and control groups was 3.6±1.7 degrees C. and 3.3±1.4 degrees C., respectively. There was a fall in temperature of the affected foot in both groups after 2 weeks with a further reduction in temperature in the active group at 4 weeks (active and placebo vs baseline; p=0.001; p=0.01, respectively), but no difference was seen between groups. An improvement in symptoms was seen in the active group compared with the placebo group (p<0.001). Reduction in bone turnover (means±SEM) was greater in the active than in the control group. Urinary deoxypyridinoline in the pamidronate treated group fell to 4.4±0.4 nmol/mmol creatinine at 4 weeks compared with 7.1±1.0 in the placebo group (p=0.01) and bone-specific alkaline phosphatase fell to 14.1±1.2 u/l compared with 18.6±1.6 u/l after 4 weeks, respectively (p=0.03). The authors concluded that the bisphosphonate, pamidronate, given as a single dose leads to a reduction in bone turnover, symptoms and disease activity in diabetic patients with active Charcot neuroarthropathyl .

Type II Diabtes and Statins

Type 2 diabetes is associated with a substantially increased risk of cardiovascular disease, but the role of lipid-lowering therapy with statins for the primary prevention of cardiovascular disease in diabetes is inadequately defined. One study aimed to assess the effectiveness of atorvastatin 10 mg daily for primary prevention of major cardiovascular events in patients with type 2 diabetes without high concentrations of LD-cholesterol. 2838 patients aged 40-75 years in 132 centers in the UK and Ireland were randomized to placebo (n=1410) or atorvastatin 10 mg daily (n=1428). Study entrants had no documented previous history of cardiovascular disease, an LDL-cholesterol concentration of 4.14 mmol/L or lower, a fasting triglyceride amount of 6.78 mmol/L or less, and at least one of the following: retinopathy, albuminuria, current smoking, or hypertension. The primary endpoint was time to first occurrence of the following: acute coronary heart disease events, coronary re-vascularisation, or stroke. Analysis was by intention to treat. The trial was terminated 2 years earlier than expected because the pre-specified early stopping rule for efficacy had been met. Median duration of follow-up was 3.9 years (IQR 3.0-4.7). 127 patients allocated placebo (2.46 per 100 person-years at risk) and 83 allocated atorvastatin (1.54 per 100 person-years at risk) had at least one major cardiovascular event (rate reduction 37% [95% CI −52 to −17], p=0.001). Treatment would be expected to prevent at least 37 major vascular events per 1000 such people treated for 4 years. Assessed separately, acute coronary heart disease events were reduced by 36% (−55 to −9), coronary re-vascularisations by 31% (−59 to 16), and rate of stroke by 48% (−69 to −11). Atorvastatin reduced the death rate by 27% (−48 to 1, p=0.059). No excess of adverse events was noted in the atorvastatin group. The study authors determined that Atorvastatin 10 mg daily is safe and efficacious in reducing the risk of first cardiovascular disease events, including stroke, in patients with type 2 diabetes without high LDL-cholesterol. The researchers stated that no justification is available for having a particular threshold level of LDL-cholesterol as the sole arbiter of which patients with type 2 diabetes should receive statins. The authors concluded that debate about whether all people with this disorder warrant statin treatment should now focus on whether any patients are at sufficiently low risk for this treatment to be withheld¹⁰¹.

Osteoporosis and Interleukin 6

Osteoporosis is a condition that is common with aging and especially in post-menopausal women. The etiology has often been ascribed to abnormalities in calcium metabolism. However many patients with osteopenia/osteoporosis have in common pain and inflammation. Sota Omoigui's Law of Pain states that the origin of all pain syndromes is inflammation and the inflammatory response¹. Many patients with various inflammatory pain syndromes have osteopenia/osteoporosis as an accompanying feature. A notable example is the osteoporosis presenting commonly in Complex regional pain syndrome/Reflex sympathetic dystrophy (CRPS-I/RSD)¹⁰². Interleukin-6 plays a significant role in immunology and inflammation. In addition, it has been shown to contribute to the process of bone remodeling. This it does by stimulating osteoclastogenesis and osteoclast activity⁵. In addition to increasing osteoclast numbers, IL-6 has been shown to stimulate bone resorption in rat long bones⁷. Furthermore, elevated levels of Interleukin-6 have been observed in conditions of rapid skeletal turnover and hypercalcemia as in Paget's disease and multiple myeloma^{8, 9}. In multiple myeloma, radiologic examinations reveals osteolytic lesion and the most common finding is diffuse osteopenia¹⁰. Adhesion of multiple myeloma cells to stromal cells triggers IL-6 secretion by the stromal cells¹¹. This results in increased osteoclastic activity that in turn results in osteoporosis, painful osteolytic lesions and hypercalcemia characteristic of multiple myeloma⁹. In their youth, women are protected from osteoporosis because of the presence of sufficient levels of estrogen. Estrogen blocks the osteoblast's synthesis of Interleukin 6. Estrogen may also antagonize the interleukin 6 receptors. Decline in estrogen production is often associated with osteopenia/osteoporosis in postmenopausal women. Estrogen's ability to repress IL-6 expression was first recognized in human endometrial stromal cells¹³. Additional clues came from the observations that menopause or ovariectomy resulted in increased IL-6 serum levels¹⁴, increased IL-6 mRNA levels in bone cells¹⁵, and increased IL-6 secretion by mononuclear cells^{16 17 18}. Further evidence for estrogen's ability to repress IL-6 expression is derived from studies, which demonstrated that estradiol inhibits bone marrow stromal cell and osteoblastic cell IL-6 protein and mRNA production in vitro¹⁹ and that estradiol was as effective as neutralizing antibody to IL6 in suppressing osteoclast development in murine bone cell cultures¹⁹ or in ovariectomized mice²⁰. In one study, the efficacy of etidronate with respect to osteoporosis, inhibition of bone resorption and destruction, and anti-inflammation in patients with rheumatoid arthritis (RA) was examined for 72 weeks. Sixty-three patients with RA (56 women, 7 men) were divided into a group that received intermittent cyclical etidronate therapy (ICET) (31 patients) and a non-ICET group (32 patients). Over a 72 week followup period, the urinary deoxypyridinoline (DPD), serum bone alkaline phosphatase (BAP), bone mineral density (BMD), Larsen damage score, lansbury activity index, and concentrations of serum C-reactive protein (CRP) and serum interleukin 6 (IL6) of the 2 groups were compared. In the non-ICET group, a significant decrease in BMD and a significant increase in the Larsen damage score were observed. In the ICET group, the level of DPD started to decrease 12 weeks after etidronate administration and progression of the Larsen damage score was significantly inhibited. IL-6 concentration was significantly decreased 72 weeks after etidronate administration. Concentrations of BAP and CRP and the Lansbury activity index were not significantly different between the ICET and the non-ICET groups. A significant correlation between the IL-6 and DPD concentrations was observed. The authors concluded that Etidronate was effective at inhibiting bone resorption and destruction in study patients with RA, while not increasing BAP concentrations; and a correlation was observed between the concentration of DPD and IL-6, indicating the anti-inflammatory effect of Etidronate¹⁰³. Another study of 7 patients with bone metastases due to solid tumors, was performed to evaluate the in vivo effects of the bisphosphonate, pamidronate, on blood levels of IL-6. Pamidronate was injected intravenously at 60 mg over 3 h. Venous blood samples were drawn before, at 1-h intervals during pamidronate infusion, then after 1 and 3 days. Mean serum levels of IL-6 significantly decreased during pamidronate,infusion, then after 1 and 3 days, IL-6 mean levels still remained lower than control level, but differences were not significant. The authors concluded that pamidronate infusion induces a rapid but transient decline in IL-6 blood concentrations¹⁰⁴. Based upon our clinical experience and inflammation research, we have proposed that a primary origin of osteoporosis is IL-6 mediated inflammation and the inflammatory response¹⁰⁵.

Osteoporosis and Bisphosphonates

Bisphosphonates are inorganic chemical compounds that bind to hydroxyapatite in bone and prevent osteoclastic absorption of bone. They directly inhibit osteoclast activity. This inhibition of osteoclast activity contributes to inhibition of bone resorption without inhibiting bone formation and mineralization³. Bisphosphonates also work to restore the critical linkage between bone destruction and formation. Furthermore, bisphosphonates appear to reverse the decrease in bone density induced by glucocorticoids often used to treat myeloma². Quite often, patients with inflammatory pain syndromes have osteopenia/osteoporosis¹⁰⁶. This is due to the inflammatory chemical mediators that are present in those pain syndromes or due to chronic steroid administration. Our research interest in bisphosphonates started with our clinical observation of the pain relief obtained after intravenous pamidronate infusions in a patient with Paget's disease despite the absence of hypercalcemia. We have subsequently observed numerous patients with various inflammatory pain syndromes with or without osteopenia/osteoporosis respond with significant relief of their pain syndromes to infusion of pamidronate¹⁰⁷. Pamidronate has also been reported to result in complete remission in a case of refractory Hypertrophic Pulmonary Osteoarthropathy in Cystic Fibrosis⁴.

Osteoporosis and Statins

3-hydroxy-3-methylglutaryl coenzyme A reductase inhibitors (statins) have been shown to stimulate bone formation in laboratory studies, both in vitro and in vivo. Statin use in most, but not all observational studies is associated with a reduced risk of fracture, particularly hip fracture, even after adjustment for the confounding effects of age, weight and other medication use. This beneficial effect has not been observed in clinical trials designed to assess cardiovascular endpoints¹⁰⁸. In one study analysis of covariance model that was adjusted for age, body mass index, race, and vitamin use, men using statin drugs were more likely to have a greater BMD of the spine (p<0.005). The mean difference (effect size) was 0.05 g/cm2 (95% confidence interval [CI] of 0.02-0.09), about 5.3% greater BMD. In women, the association was not significant. The risk of osteoporosis (defined as a T-score<or =−2.5) was determined using logistic regression analysis after adjustment for potential confounding variables. Although not statistically significant, men who received statin drugs for more than 2 yr were approximately half as likely to develop osteoporosis (odds ratio [OR]=0.55, 95% CI=0.28-1.08). A similar effect was observed in women taking statins for any length of time (OR=0.36, 95% CI=0.12-1.07). The authors of the study suggest that statin drugs may decrease osteoporosis risk, warranting a randomized controlled trial¹⁰⁹. In a cross-sectional study set in southeastern Australia, the researchers evaluated the association between statin use, fracture risk, and BMD in 1375 women (573 with incident fractures and 802 without incident fracture, all drawn from the same community). Fractures were identified radiologically. Medication use and lifestyle factors were documented by questionnaire. Unadjusted odds ratio for fracture associated with statin use was 0.40 (95% confidence interval [CI], 0.23-0.71). Adjusting for BMD at the femoral neck, spine, and whole body increased the odds ratio to 0.45 (95% CI, 0.25-0.80), 0.42 (95% CI, 0.24-0.75), and 0.43 (95% CI, 0.24-0.78), respectively. Adjusting for age, weight, concurrent medications, and lifestyle factors had no substantial effect on the odds ratio for fracture. Statin use was associated with a 3% greater adjusted BMD at the femoral neck (P=0.08), and BMD tended to be greater at the spine and whole body but did not achieve statistical significance. The researchers concluded that the substantial 60% reduction in fracture risk associated with statin use is greater than would be expected from increases in BMD alone¹¹⁰. In vitro, cerivastatin inhibits parathyroid hormone (PTH)-stimulated bone resorption. Using a panel of 40 statin analogs, which showed variable effects on HMG-CoA reductase activity, a research study found that the ability of statin compounds to inhibit bone resorption is directly related to HMG-CoA reductase activity¹¹¹. In this study, the ability of statins to inhibit bone resorption was found to be directly related to their inhibitory effect on HMG-CoA reductase activity. In the abstract of the study, the authors stated as follows: Statins, which are inhibitors of 3-hydroxy-3-glutaryl-coenzyme A (HMG-CoA) reductase, decrease the hepatic biosynthesis of cholesterol by blocking the mevalonate pathway. Nitrogen-containing bisphosphonate drugs also inhibit the mevalonate pathway, preventing the production of the isoprenoids, which consequently results in the inhibition of osteoclast formation and osteoclast function. The authors hypothesized that statins could affect bone metabolism in vivo through effects on osteoclastic bone resorption. In contrast to other studies, none of the statins tested showed anabolic effects in parietal bone explant cultures. Taken together, the authors concluded that statins inhibit bone resorption in vitro, which correlates directly with the potency of the compounds for inhibition of HMG-CoA reductase activity.

Arthris and Interleukin-6

Interleukin-1 (IL1), a cytokine produced by chondrocytes and other cells in the joint, plays an important role in cartilage degradation by stimulating the synthesis of degradative enzymes that inhibit the production of proteoglycans. Other cytokines that appear to act synergistically with IL-1 to promote matrix breakdown are tumor necrosis factor-alpha (TNF-alpha) and interleukin-6 (IL-6). During times of stress or inflammation IL-6 levels are increased. Inflammatory joint disease, particularly rheumatoid arthritis²¹, is associated with increased synovial fluid levels of IL-6²². Although Osteoarthritis has previously been considered a non-inflammatory form of arthritis, there are changes that occur within the joint that are associated with inflammation. Inflammation is aggravated by the introduction of bone and cartilage breakdown products into the synovial fluid. Cells in the synovium phagocytize these products, resulting in chronic, low-grade inflammation. Consequently, the synovial membrane becomes thickened. Inflammation of the synovial membrane may be absent in the earlier stages of Osteoarthritis; however, as the disease progresses, some degree of synovitis usually exists. Once mild synovial inflammation is established, the synovium becomes a source of cartilage-degrading enzymes (e.g., MMPs) and cytokines, including IL-1, IL-6, and TNF-alpha. These substances diffuse through the synovial fluid and cause further degradation of articular cartilage. IL-1 and TNF-alpha stimulate the chondrocytes to produce more degrading enzymes, and the process continues in a vicious cycle. IL-11, IL-6, and TNF-alpha are believed to be the main cytokines linked to the disease process¹.

Arthritis and Bisphosphonates

Pamidronate has resulted in pain reduction in patients with osteoarthritis (with and without osteoporosis) in our clinic, via its anti-inflammatory properties resulting in a subsequent reduction of bone resorption and inflammatory bone pain¹¹². The quick onset of pain relief observed in our patients can only be attributed to its anti-interleukin-6 effect. Several literature abound documenting the anti-interleukin-6 effect of bisphosphonates. Bisphosphonates inhibit the production of pro-inflammatory cytokine interleukin-6 in tumoral cell lines of human osteoblastic phenotype (MG63 and SaOs cells), and in peripheral blood mononuclear cells (PBMC)²³. Pamidronate infusion has significantly decreased the mean serum levels of Interleukin-6 in patients with advanced solid tumors and bone metastases²⁴. Pamidronate and other bisphosphonates inhibit the production by osteoblasts of the inflammatory cytokine interleukin-6, a growth factor essential to myeloma cells . In patients with Paget's disease of bone, bisphosphonate therapy is associated with a significant reduction of Interleukin-6 soluble receptor (sIL-6R) serum levels²⁶. Bisphosphonates also inhibit IL-1 and TNF-alpha stimulated IL-6 release in cultures of human osteoblastic osteosarcoma cells²⁷. Osteoblasts exposed to small amounts of bisphosphonate elaborate a soluble inhibitor, which interferes with osteoclast formation and development²⁸. Furthermore, bisphosphonates prevent apoptosis of murine osteocytic MLO-Y4 cells, whether it is induced by etoposide, TNF-alpha, or the glucocorticoid dexamethasone²⁹. In a recent study appearing in the journal, Clinical & Experimental Rheumatology Masuda-Aiba et al observed that a new third-generation bisphosphonate, YM529, represents a candidate treatment for arthritis¹¹³. The authors report that prophylactic or therapeutic treatment of animals with experimental arthritis with YM529 suppressed the severity of disease and suggest that YM529 may act on arthritic joints locally to prevent inflammation. These data are consistent with previous clinical studies investigating the efficacy of other bisphosphonates in patients with rheumatoid arthritis. The authors concluded that although further experiments are necessary to elucidate the underlying mechanisms, YM529 deserves consideration as a treatment for this disease.

Arthritis and Statins

MMP-9 or Gelatinase B, a member of the matrix metalloproteinase family (MMPs), plays important roles in physiological events such as tissue remodeling and in pathological processes that lead to destructive bone diseases, including osteoarthritis and periodontitis. In addition to its effect on the increase of total bone mass, statin (an HMG-CoA reductase inhibitor) suppresses the expression of MMPs. In this study, the researchers proposed that simvastatin reduces MMP-9 expression in osteoblasts and HT1080 fibrosarcoma cell line. Gelatin zymography, Western blot analysis and reverse transcriptase-PCR were used to investigate the effects of simvastatin on MMP-9 in primary calvaria cells, U2-OS osteosarcoma cells, and HT1080 fibrosarcoma cells. The results from gelatin zymography and Western blot analysis revealed that simvastatin suppressed MMP-9 activity in these cells in concentration- and time-dependent manners. The effective concentrations of simvastatin were 100-500 nM, 5-15 microM, and 2.5-10 microM in primary calvaria, U2-OS, and HT1080 cells, respectively. The authors concluded that collectively, these results suggest that simvastatin is a potent drug for inhibition of MMP-9 expression in osteoblastic cells and HT1080 fibrosarcoma cells¹¹⁴. In another study, the researchers postulated that 3-Hydroxy-3-methylglutaryl-CoA reductase inhibitors (statins) exert favorable effects on lipoprotein metabolism, but may also possess anti-inflammatory properties. The authors explored the activities of simvastatin, a lipophilic statin, in a Th1-driven model of murine inflammatory arthritis. They reported in this study that simvastatin markedly inhibited not only developing but also clinically evident collagen-induced arthritis in doses that were unable to significantly alter cholesterol. concentrations in vivo. Ex vivo analysis demonstrated significant suppression of collagen-specific Th1 humoral and cellular immune responses. Moreover, simvastatin reduced anti-CD3/anti-CD28 proliferation and IFN-gamma release from mononuclear cells derived from peripheral blood and synovial fluid. Proinflammatory cytokine production in vitro by T cell contact-activated macrophages was suppressed by simvastatin, suggesting that such observations have direct clinical relevance. The authors concluded that these data clearly illustrate the therapeutic potential of statin-sensitive pathways in inflammatory arthritis¹¹⁵. In one study, the authors set out to clarify whether the inhibition of sterol or nonsterol derivatives arising from mevalonate biotransformation plays a major role in the in vivo anti-inflammatory action of statins¹¹⁶. Hepatic synthesis of all these derivatives was inhibited in mice by administered statins, whereas squalestatin inhibited only sterol derivatives. Using a short-term treatment schedule, the authors found that statins reduced the hepatic activity of 3-hydroxy-3-methylglutaryl coenzyme A reductase without affecting blood cholesterol. This treatment inhibited lipopolysaccharide- and carrageenan-induced pouch leukocyte recruitment and the exudate production of interleukin-6, monocyte chemotactic protein-1, and RANTES. Coadministration of mevalonate reversed the effect of statin on leukocyte recruitment. The inhibition of sterol synthesis by squalestatin did not have any anti-inflammatory effect, indicating that the biosynthesis of nonsterol compounds arising from mevalonate is crucial for the in vivo regulation of cytokine and chemokine production by statins. The authors concluded that inhibition by statins may account for the reported anti-inflammatory effects of these drugs and may provide a biochemical basis for the recently reported effects of statins in the prevention of cardiovascular disease and mortality.

Dementia, Alzheimer's Disease and Interleukin 6

Vascular (formerly Arteriosclerotic) Dementia (MID, Multi-infarct dementia) is characterized by a history of transient ischemic attacks with brief impairment of consciousness, fleeting pareses, or visual loss. The dementia may also follow a succession of acute cerebrovascular accidents or, less commonly, a single major stroke. Some impairment of memory and thinking then becomes apparent. Onset, which is usually in later life, can be abrupt, following one particular ischemic episode, or there may be more gradual emergence. The dementia is usually the result of infarction of the brain due to vascular diseases, including hypertensive cerebrovascular disease. The infarcts are usually small but cumulative in their effect. Vascular dementia can occur with other types of dementia such as Alzheimer's disease. Compared with Alzheimer's disease, vascular dementia can affect distinct parts of the brain and particular abilities may remain relatively unaffected. Alzheimer's disease affects the entire brain. Symptoms of vascular dementia remain steady for a while and then suddenly decline. In Alzheimer's disease the decline is more constant.

Alzheimer's disease (AD), the most common form of dementia, is a progressive, degenerative disorder of the central nervous system. Interleukin 6 mediated inflammation play a role in several age-related diseases, including Alzheimer's disease. The Health, Aging and Body Composition Study¹¹⁷ enrolled 3,031 black and white men and women, with an average age of 74. The researchers took blood levels of interleukin-6 (ILR6), C-reactive protein and tumor necrosis factor and then repeated the tests two years later. A battery of mental tests was also given to evaluate concentration, memory, language and other measures of cognitive functioning, both at the start and two years later. After adjusting for age and other factors, they found that those who had the highest levels of inflammation—whose blood levels of IL-6 and C-reactive protein were in the highest one-third—had more cognitive decline compared to those whose blood levels of those substances were in the lower third. If their IL-6 result was high, they were 34 percent more likely to have cognitive decline than those whose scores on the tests were in the lower third. If their C-reactive protein levels were in the top third, they were 41 percent more likely to have cognitive decline than those in the lower third. Although those who suffered cognitive decline also had higher levels of tumor necrosis factor, the differences weren't statistically significant. The study found no relationship between the use of non-steroidal anti-inflammatory drugs (NSAIDs) and inflammation levels. This is not surprising as NSAIDs inhibit cyclooxygenase and affect prostaglandin synthesis but have no effect on IL-6 inflammation.

Dementia, Alzheimer's Disease and Statins

Increased circulating cholesterol has been long linked to an increased risk of coronary artery disease (CAD), and is now linked to an increased risk of developing Alzheimer s disease (AD). The neuropathologic link between CAD and AD manifests as increased incidence of cerebral senile plaques in both disorders. In one study, the researchers showed that AD-like neuropathology occurred in the brains of cholesterol-fed rabbits; including increased beta-amyloid (A beta)¹¹⁸. The major hallmarks of AD include selective neuronal cell death and the presence of amyloid deposits and neurofibrillary tangles. Apolipoprotein E (ApoE) has also been shown to co-localize with these neuropathological lesions. Putative pathological functions or “risk-factor activities” of ApoE-epsilon4 include its role in promoting amyloid accumulation, neurotoxicity, oxidative stress and neuro fibrillary tangles. ApoE has been shown essential for amyloid beta-peptide fibrillogenesis and deposition, a defining pathological feature of this disease. The human ApoE gene has three alleles (epsilon2, epsilon3, epsilon4)-all products of the same gene. The epsilon3-allele accounts for the majority of the ApoE gene pool (approximately 70-80%), the epsilon4-allele accounts for 10-15% and the epsilon2 allele for 5-10%. Inheritance of the epsilon4-allele strongly increases the risk for developing AD at an earlier age. ApoE mRNA is most abundant in the liver followed by the brain, where it is synthesized and secreted primarily by astrocytes. ApoE protein and mRNA are further detected in cortical and hippocampal neurons in humans. ApoE gene expression is induced by brain injury in some neurons and upregulated in astrocytes during aging. In AD, an increased ApoE mRNA was reported in the hippocampus. The risk for AD has been reported to correlate with transcriptional activity of the ApoE gene. Binding sites for putative transcriptional factors (TF), such as AP-1, AP-2 and NF-kappaB, are present in the ApoE promoter. The promoter also contains sites for the inflammatory response transcription factors IL-6 RE-BP, MED1, STAT1 and STAT2¹¹⁹.

Because astrocytes and microglia represent the major source of extracellular apoE in brain, one study investigated apoE secretion by glia. The authors determined that protein prenylation is required for apoE release from a continuous microglial cell line, primary mixed glia, and from organotypic hippocampal cultures. Using selective protein prenylation inhibitors, apoE secretion was found to require protein geranylgeranylation. This prenylation involved a protein critical to apoE secretion, not apoE proper. ApoE secretion could also be suppressed by inhibiting synthesis of mevalonate, the precursor to both types of protein prenylation, using hydroxyl-3-methylglutaryl coenzyme A reductase inhibitors (statins). The authors stated that recent reports have described the beneficial effects of statins on the risk of dementia. The authors further stated that their finding that protein geranylgeranylation is required for apoE secretion in the brain parenchyma provides another contributing mechanism to explain the effective properties of statins against the development of dementia. They concluded that in this model, statin-mediated inhibition of mevalonate synthesis, an essential reaction in forming geranylgeranyl lipid, would lower extracellular levels of parenchymal apoE. Because apoE has been found necessary for plaque development in transgenic models of Alzheimer's disease, suppressing apoE secretion by statins could reduce plaques and, in turn, improve cognitive function¹²⁰.

Statins have been reported to mediate changes in neuronal survival and cytoskeleton, including the microtubule-associated protein tau, a major constituent of the tangles. In one study to determine the effect of statin on the cytoskeleton, the authors challenged rat primary neuron cultures by lovastatin and determined the metabolite that is critical for structural integrity and survival of neurons. During the blockade of 3-hydroxy-3-methylglutaryl-coenzyme A reductase, the neuritic plaque was affected and eventually was completely destroyed. This process was not part of the execution phase of apoptosis and was marked by alterations in the microfilament and microtubule system. The distribution and phosphorylation of protein tau changed. Immunoblot analysis and indirect immunofluorescence revealed a transient increase in tau phosphorylation, which ceased during the execution of apoptosis. The researchers determined that all of these effects could be linked to the lack of the geranylgeranylpyrophosphate intermediate. Inhibition of the geranylgeranylation of Rho family GTPases (geranylgeranyl-transferase I) evoked similar changes in neurons. The researchers stated that these data and their findings that statin treatment reduced the membrane-bound fraction of RhoA-GTPase in neurons suggest that reduced levels of functional small G proteins are responsible for the observed effects. They concluded that their data demonstrates that lovastatin concentrations that are able to suppress not only cholesterol but also geranylgeranylpyrophosphate formation may evoke phosphorylation of tau reminiscent of preclinical early stages of Alzheimer's disease and, when prolonged, apoptosis^{121.}

An observational study of 1037 postmenopausal women with coronary heart disease enrolled in the Heart and Estrogen/progestin Replacement Study (participants at 10 of 20 centers), was undertaken to determine whether serum lipoprotein levels, the 4-year change in serum lipoprotein levels, and the use of statin drugs are associated with cognition in older women without dementia. The Modified Mini-Mental State Examination was administered at the end of the study after 4 years of follow-up. Women whose score was less than 84 points (>1.5 SDs below the mean) were classified as having cognitive impairment. Lipoprotein levels (total, high-density lipoprotein, and low-density lipoprotein [LDL] cholesterol and triglycerides) were measured at baseline and at the end of the study; statin use was documented at each visit. Compared with women in the lower quartiles, women in the highest LDL cholesterol quartile at cognitive testing had worse mean plus minus SD Modified Mini-Mental State Examination scores (93.7 plus minus 6.0 vs 91.9 plus minus 7.6; P=0.002) and an increased likelihood of cognitive impairment (adjusted odds ratio, 1.76; 95% confidence interval, 1.04-2.97). A reduction in the LDL cholesterol level during the 4 years tended to be associated with lower odds of impairment (adjusted odds ratio, 0.61; 95% confidence interval, 0.36-1.03) compared with women whose levels increased. Higher total and LDL cholesterol levels, corrected for lipoprotein(a) levels, were also associated with a worse Modified Mini-Mental State Examination score and a higher likelihood of impairment, whereas high-density lipoprotein cholesterol and triglyceride levels were not associated with cognition. Compared with nonusers, statin users had higher mean plus minus SD Modified Mini-Mental State Examination scores (92.7 plus minus 7.1 vs 93.7 plus minus 6.1; P=0.02) and a trend for a lower likelihood of cognitive impairment (odds ratio, 0.67; 95% confidence interval, 0.42-1.05), findings that seemed to be independent of lipid levels. The authors concluded that high LDL and total cholesterol levels are associated with cognitive impairment, and lowering these lipoprotein levels may be a strategy for preventing impairment¹²². Another study examined the association between the use of lipid-lowering agents (ILAs) and dementia, adjusting for other markers of health, and investigated factors associated with LLA use. The authors performed a cohort study of LLA use and a case-control study of dementia in relation to LIA use, in a secondary analysis of the Canadian Study of Health and Aging( a nationally representative population-based survey of Canadians 65 years and older). To examine features associated with statin use, the authors evaluated data on 2305 people for whom health information, drug use, and cognitive status were known. To examine the relationship between LLA use and dementia, the authors selected incident cases of dementia (n=492, of whom 326 had Alzheimer disease) that occurred between the first and second waves of the study. Control subjects were 823 persons examined during the first and second phases of the Canadian Study of Health and Aging who had no cognitive impairment. Results from the study showed that use of LLAs was significantly (P<0.001) more common in younger (65-79 years) than in older (>or =80 years) people. It was not associated with other factors indicating a healthy lifestyle, but was associated with a history of smoking and hypertension. Use of statins and other LLAs reduced the risk of Alzheimer disease in subjects younger than 80 years, an effect that persisted after adjustment for sex, educational level, and self-rated health (odds ratio, 0.26; 95% confidence interval, 0.08-0.88). There was no significant effect in subjects 80 years and older. The researchers concluded that while the possibility of indication bias in the original observations cannot be excluded, it was not demonstrated in LLA use in this study. Lipid-lowering agent use was associated with a lower risk of dementia, and specifically of Alzheimer disease, in those younger than 80 years²³.

Dementia, Alzheimer's Disease and Bisphosphonates

There is very little literature on the use of bisphosphonates in patients with dementia or Alzheimer's disease. In a clinical case report of primary hyperparathyroidism in an 89-year-old woman causing profound neuropsychiatric symptoms, the use of bisphosphonate therapy led to marked but temporary improvements in her mental state¹²⁴. Considering the role of Cholesterol in atherosclerosis, vascular dementia and Alzheimer's disease, bisphosphonates should play a future role in the prevention and treatment of dementia and Alzheimer's disease.

Hypertension and Interleukin 6

IL-6 is elevated in plasma of preeclamptic women, and twofold elevation of plasma IL-6 increases vascular resistance and arterial pressure in pregnant rats, suggesting a role of the cytokine in hypertension of pregnancy. In one study, the authors tested the hypothesis that IL-6 directly impairs endothelium-dependent relaxation and enhances vascular contraction in systemic vessels of pregnant rats¹²⁵. Active stress was measured in aortic strips isolated from virgin and late pregnant Sprague-Dawley rats and then nontreated or treated for 1 h with IL-6 (10 pg/ml to 10 ng/ml). In endothelium-intact vascular strips, phenylephrine (Phe, 10⁻⁵ M) caused an increase in active stress that was smaller in pregnant (4.2±0.3) than virgin rats (5.1±0.3×10⁴ N/m²). IL-6 (1,000 pg/ml) caused enhancement of Phe contraction that was greater in pregnant (10.6±0.7) than virgin rats (7.5±0.4×10⁴ N/M²). The authors concluded that IL6 inhibits endothelium-dependent NO-cGMP-mediated relaxation and enhances contraction in systemic vessels of virgin and pregnant rats. The greater IL-6-induced inhibition of vascular relaxation and enhancement of contraction in systemic vessels of pregnant rats supports a direct role for IL-6 as one possible mediator of the increased vascular resistance associated with hypertension of pregnancy.

Statins and Hypertension

Recent studies have shown that short-term use of statins can reduce blood pressure (BP) significantly. To determine the long-term effects of statins on BP and aortic stiffness, a single-blind randomized prospective study was performed on 85 hyperlipidemic hypertensive patients whose BP was insufficiently controlled by antihypertensive therapy. Every 3 months, aortic stiffness was assessed by measuring pulse wave velocity (PWV). Patients were randomly allocated to groups treated with pravastatin, simvastatin, fluvastatin, or a nonstatin antihyperlipidemic drug. No significant differences in patient characteristics, kinds of antihypertensive drugs, BP, ankle brachial index, PWV, or serum lipid, creatinine, or C-reactive protein levels were found between the four groups at the start of the study. During the 12-month treatment period, PWV did not change in the pravastatin group or nonstatin group, but it was transiently reduced in the simvastatin group and significantly decreased in the fluvastatin group, even though the doses of the statins used in this study were lower than the usually prescribed dose. All four antihyperlipidemic drugs significantly decreased serum cholesterol levels without affecting BP, ankle brachial index, or serum triglyceride levels. The C-reactive protein serum levels decreased significantly in the three statin groups but not in the nonstatin group. The authors concluded that these results suggest that long-term use of fluvastatin by hyperlipidemic hypertensive patients is associated with a significant reduction in aortic stiffness without any effect on BP¹²⁶. Other studies have suggested that lipid-lowering strategies, and particularly statins, could influence blood pressure (BP) control. The aim of the one study was to evaluate the effect of different lipid-lowering strategies on BP control of subjects with hypercholesterolemia who were enrolled in the prospective, population-based, longitudinal Brisighella Heart Study. A total of 1356 subjects with total cholesterol levels>or=239 mg/dL were randomly treated for 5 years (1988-1993) with 1 of these lipid-lowering regimens: low-fat diet, cholestyramine, gemfibrozil, or simvastatin. Participants were divided at baseline into 4 quartiles according to systolic BP level and examined for the percent change in systolic and diastolic BP during the 5 years of treatment. In the study results, a significant decrease in BP was observed in the 2 upper quartiles of systolic BP (>or=140 mm Hg) and was greater in subjects treated with cholesterol-lowering drugs who also had a greater reduction in plasma levels of low-density lipoprotein cholesterol. The BP decrease was greater in patients treated with statin drugs and, among those treated with anti-hypertensive drugs, in subjects in the fourth quartile. The authors concluded that the use of lipid-lowering measures could significantly improve BP control in subjects with both hypercholesterolemia and hypertension. The authors further stated that reduction in BP seems to be enhanced in subjects treated with statins

Bisphosphonates and Hypertension

There is very little literature on the use of bisphosphonates in patients with hypertension. Considering the role of Cholesterol in atherosclerosis, bisphosphonates should play a future role in the prevention and treatment of hypertension.

Age-Related Disorders and Interleukin 6

Overproduction of IL-6, a pro-inflammatory cytokine, is associated with a spectrum of age-related conditions including cardiovascular disease, osteoporosis, arthritis, type 2 diabetes, certain cancers, periodontal disease, frailty, and functional decline. To describe the pattern of change in IL-6 over 6 years among older adults undergoing a chronic stressor, this longitudinal community study assessed the relationship between chronic stress and IL-6 production in 119 men and women who were caregiving for a spouse with dementia and 106 noncaregivers, with a mean age at study entry of 70.58 (SD=8.03) for the full sample. On entry into this portion of the longitudinal study, 28 of the caregivers' spouses had already died, and an additional 50 of the 119 spouses died during the 6 years of this study. Levels of IL-6 and health behaviors associated with IL-6 were measured across 6 years. Caregivers' average rate of increase in IL-6 was about four times as large as that of noncaregivers. Moreover, the mean annual changes in IL-6 among former caregivers did not differ from that of current caregivers even several years after the death of the impaired spouse. There were no systematic group differences in chronic health problems, medications, or health-relevant behaviors that might have accounted for caregivers' steeper IL-6 slope. These data provide evidence of a key mechanism through which chronic stressors may accelerate risk of a host of age-related diseases by prematurely aging the immune response.¹²⁸

Age-Related Disorders and Statins

Considering the role of Cholesterol in age-related disorders such as osteoporosis, arthritis, type 2 diabetes, dementia and Alzheimer's disease, statins should play a future role in the prevention and treatment of age-related disorders.

Age-Related Disorders and Bisphosphonates

Considering the role of Cholesterol in age-related disorders such as osteoporosis, arthritis, type 2 diabetes, dementia and Alzheimer's disease, bisphosphonates should play a future role in the prevention and treatment of age-related disorders.

Conclusion

Cholesterol Synthesis is the trigger and Interleukin 6 mediated inflammation is the common causative factor and therapeutic target for atherosclerosis, peripheral vascular disease, coronary artery disease, and age-related disorders including osteoporosis, arthritis, type 2 diabetes, dementia and Alzheimer's disease.

Isoprenoids, which are intermediates, generated in the cholesterol biosynthesis pathway, play a more significant role than the end product cholesterol, in activation of Interleukin 6 mediated inflammation. Isoprenoids are generated by endogenous cellular cholesterol synthesis in the body as well as by cholesterol synthesis in activated monocytes during the inflammatory response. Isoprenoids are an integral component of the signaling pathway for Interleukin 6 mediated inflammation.

Statins and Bisphosphonates have similar mechanisms of action and act on similar diseases in the following ways:

1. Statins and Bisphosphonates inhibit the Mevalonate to Cholesterol conversion pathway and cause isoprenoid depletion.

2. Statins and Bisphosphonates inhibit the signaling pathway for Interleukin 6 mediated inflammation by isoprenoid depletion.

3. Statins and Bisphosphonates inhibit bone resorption.

4. Treatment with statins, bisphosphonates and/or a cholesterol lowering agent or technique will be the most effective method of prevention and treatment of atherosclerosis, peripheral vascular disease, coronary artery disease, and age-related disorders including osteoporosis, arthritis, type 2 diabetes, dementia and Alzheimer's disease.

It will be apparent to those skilled in the art that variations and modifications to the specific embodiments disclosed herein may be made without departing from the scope of the invention.

### REFERENCES

¹Am J Cardiol 2000; 85:10A-17A.

Rationale and design of the Cardiac Hospitalization Atherosclerosis Management Program at the University of California Los Angeles.

Fonarow G C, Gawlinski A.

²JAMA. 2003 Aug. 20;290(7):932-40

Emerging risk factors for atherosclerotic vascular disease: a critical review of the evidence.

Hackam D G, Anand S S.

³Lancet 1997;349:1436-42.

Global mortality, disability, and the contribution of risk factors: global burden of disease study.

Murray C J, Lopez A D.

⁴N Engl J Med 1999;340:115-26

Atherosclerosis an inflammatory disease.

Ross R.

⁵Circulation 2002;105:1135-43.

Inflammation and atherosclerosis.

Libby P, Ridker P M, Maser A.

⁶J. Clin. Invest. 110:905-911 (2002). doi:10.1172/JCI200216452.

Consequences of cellular cholesterol accumulation: basic concepts and physiological implications

Tabas I.

⁷J. Lipid Res. 34:1637-1659.

Role of the liver in the maintenance of cholesterol and low density lipoprotein homeostasis in different animal species, including humans.

Dietschy, J. M., Turley, S. D., and Spady, D. K. 1993.

⁸In Molecular basis of heart disease. K. R. Chien, editor. W. B. Saunders Co. New York, New York, USA.

428-457

Lipoprotein receptors and cellular cholesterol metabolism in health and disease.

Tabas, I., and Kreiger, M. 1999.

⁹J Biol Chem. 1993;268:8569-8573.

Cholesterol loading of macrophages leads to marked enhancement of native lipoprotein(a) and apoprotein(a) internalization and degradation.

Bottalico L A, Keesler G A, Fless G M, Tabas I.

¹⁰Arteriosclerosis. 1987;7:191-196.

Modification of very low density lipoproteins leads to macrophage scavenger receptor uptake and cholesteryl ester deposition.

Mazzone T, Lopez C, Bergstraesser L.

¹¹Proc Natl Acad Sci USA. 1987;84:2995-2298

Oxidatively modified low density lipoproteins: a potential role in recruitment and retention of monocyte/macrophages during atherogenesis.

Quinn M T, Parthasarathy S, Fong L G, Steinberg D.

¹²J Clin Invest. 1991;88:1785-1792

Role of oxidized low density lipoprotein in atherogenesis.

Witztum J L, Steinberg D.

¹³Nature. 1993;362:801-809.

The pathogenesis of atherosclerosis: a perspective for the 1990s.

Ross R.

¹⁴Science. 267:1828-1831.

Role of steroidogenic acute regulatory protein in adrenal and gonadal steroidogenesis.

Lin, D., et al. 1995.

¹⁵J. Lipid Res. 34:1637-1659.

Role of the liver in the maintenance of cholesterol and low density lipoprotein homeostasis in different animal species, including humans.

Dietschy, J. M., Turley, S. D., and Spady, D. K. 1993.

¹⁶J. Clin. Invest. 110:899-904. doi:10.1172/JCI200216391.

2002. Regulation and mechanisms of macrophage cholesterol efflux.

Tall, A. R., Costet, P., and Wang, N.

¹⁷Cell. 2001 Feb. 23;104(4):503-16

Atherosclerosis: the road ahead

Glass, C. K., and Witztum, J. L.

¹⁸J. Clin. Invest. 109:1125-1131. doi: 10.1172/JCI200215593.

2002. SREBPs: activators of the complete program of cholesterol and fatty acid synthesis in the liver.

Horton, J. D., Goldstein, J. L., and Brown, M. S.

¹⁹Chang, T. Y., et al. 2001.Roles of acyl-coenzyme A:cholesterol acyltransferase-1 and -2. ²⁰Curr. Opin. Lipidol. 12:289-296

2001. Roles of acyl-coenzyme A:cholesterol acyltransferase-1 and -2.

Chang, T. Y., et al.

²¹J. Clin. Invest. 110:899-904. doi:10.1172/JCI200216391

2002. Regulation and mechanisms of macrophage cholesterol efflux.

Tall, A. R., Costet, P., and Wang, N.

²²J. Clin. Invest. 110:725-730. doi:10.1172/JCI200216388.

2002. Do oxysterols control cholesterol homeostasis?

Björkhem, I.

²³In Molecular basis of heart disease.

1999. Lipoprotein receptors and cellular cholesterol metabolism in health and disease.

Tabas, I., and Kreiger, M.

²⁴Trends Cardiovasc. Med. 7:256-263

Free cholesterol-induced cytotoxicity. A possible contributing factor to macrophage foam cell necrosis in advanced atherosclerotic lesions.

Tabas, I. 1997.

²⁵Annu. Rev. Physiol. 57:791-804.

Cell biology of atherosclerosis.

Ross, R. 1995.

²⁶Trends Cardiovasc. Med. 7:256-263

Free cholesterol-induced cytotoxicity. A possible contributing factor to macrophage foam cell necrosis in advanced atherosclerotic lesions.

Tabas, I. 1997.

²⁷Arterioscler. Thromb. Vasc. Biol. 18:1519-1522.

Apoptosis in the atherosclerotic plaque: quantitative and qualitative aspects.

Kockx, M. M. 1998.

²⁸Curr. Opin. Lipidol. 7:324-329.

1996. Cell death in atherosclerotic plaques.

Mitchinson, M. J., Hardwick, S. J., and Bennett, M. R.

²⁹Biochem J 1990;265:621-636

Interleukin-6 and the acute phase response

Heinrich P C, Castell J V, Andus T.

³⁰Adv Intern Med 1992;37:313-336

C-reactive protein and the acute phase response.

Ballou S P, Kushner I.

³¹Arch Pathol Lab Med 1987;111:265-269

C-reactive protein immunohistochemical localization in normal and atherosclerotic human aortas.

Reynolds G D, Vance R P.

³²Pathol Int 1995;45:635-641

Immuno-histochemical localization of C-reactive protein-binding sites in human atherosclerotic aortic lesions by a modified streptavidinbiotin-staining method.

Hatanaka K, Li X A, Masuda K, et al.

³³In: Weatherall D J, Ledingham J G G, Warrell D A, eds. Oxford Textbook of Medicine. New York, N.Y.: Oxford University Press; 1996:1527-1533

The acute phase response and C-reactive protein.

Pepys M B.

³⁴Circulation 1996;94:874-877

Elevated levels of interleukin-6 in unstable angina.

Biasucci L M, Vitelli A, Liuzzo G, et al.

³⁵Circulation 1999;99:2079-2084

Increasing levels of interleukin (IL)-1Ra and IL-6 during the first 2 days of hospitalization in unstable angina are associated with increasing risk of in-hospital coronary events.

Biasucci L M, Liuzzo G, Fantuzzi G, et al.

³⁶Agents Actions 1994;42:159-162

Human atherosclerotic abdominal aortic aneurysms produce interleukin (IL)-6 and interferon-gamma but not IL-2 and IL-4: the possible role for IL-6 and interferon-gamma in vascular inflammation.

Szekanecz Z, Shah M R, Pearce W H, Koch A E.

³⁷Circulation 2000;101:2149-2153

Elevation of tumor necrosis factor-alpha and increased risk of recurrent coronary events after myocardial infarction.

Ridker P M, Rifai N, Pfeifer M, et al.

³⁸Circulation 2000;101:1767-1772

Plasma concentration of interleukin-6 and the risk of future myocardial infarction among apparently healthy men.

Ridker P M, Rifai N, Stampfer M J, Hennekens C H.

³⁹J Am Coll Cardiol 1999;33:1347-1352

A prospective study of fibrinogen and risk of myocardial infarction in the Physicians' Health Study.

Ma J, Hennekens C H, Ridker P M, Stampfer M J.

⁴⁰Circulation 2000;101:1767-1772

Plasma concentration of interleukin-6 and the risk of future myocardial infarction among apparently healthy men.

Ridker P M, Rifai N, Stampfer M J, Hennekens C H.

⁴¹. Am J Cardiol 1990;65:168-172.

Elevation of C-reactive protein in “active” coronary artery disease.

Berk B C, Weintraub W S, Alexander R W

⁴²J Clin Epidemiol 2000;53:95-102

Serum C-reactive protein and fibrinogen concentrations and self-reported angina pectoris and myocardial infarction: findings from National Health and Nutrition Examination Survey III.

Ford E S, Giles W H.

⁴³Med Clin North Am 2000;84:149-161

C-reactive protein, inflammation, and coronary risk.

Morrow D A, Ridker P M.

⁴⁴Circulation 1999;100:230-235

Long-term effects of pravastatin on plasma concentration of C-reactive protein. The Cholesterol and Recurrent Events (CARE) Investigators.

Ridker P M, Rifai N, Pfeifer M A, et al.

⁴⁵Circulation 1998;97:425-428

Plasma concentration of C-reactive protein and risk of developing peripheral vascular disease.

Ridker P M, Cushman M, Stampfer M J, et al.

⁴⁶Ann Epidemiol 1999;9:166-171

High levels of plasma C-reactive protein and future risk of age-related cataract.

Schaumberg D A, Ridker P M, Glynn R J, et al.

⁴⁷Circulation 1998;97:425-428

Plasma concentration of C-reactive protein and risk of developing peripheral vascular disease.

Ridker P M, Cushman M, Stampfer M J, et al.

⁴⁸N Engl J Med 2000;342:836-843

C-reactive protein and other markers of inflammation in the prediction of cardiovascular disease in women.

Ridker P M, Hennekens C H, Buring J E, Rifai N.

⁴⁹Circulation. 2003 Nov. 11;108(19):2317-22.

Inflammatory markers and onset of cardiovascular events: results from the Health ABC study.

Cesari M, Penninx B W, Newman A B, Kritchevsky S B, Nicklas B J, Sutton-Tyrrell K, Rubin S M, Ding J, Simonsick E M, Harris T B, Pahor M

⁵⁰ Am J Cardiol. 2003 Sep. 1;92(5):522-8.

Inflammatory markers and cardiovascular disease (The Health, Aging and Body Composition [Health ABC] Study).

Cesari M, Penninx B W, Newman A B, Kritchevsky S B, Nicklas B J, Sutton-Tyrrell K, Tracy R P, Rubin S M, Harris T B, Pahor M

⁵¹Indiana University School of Medicine,

Medical Biochemistry Course Guide, 2004

Cholesterol and Bile Metabolism

King M. W.

⁵²Indiana University School of Medicine,

Medical Biochemistry Course Guide, 2004

Cholesterol and Bile Metabolism

King M. W

⁵³CMAJ.OCT. 2, 2001; 165 (7)

Statins and the prevention of dementia

Das U. N.

⁵⁴Cell1999; 98: 9-12

New insights into the interaction of Ras with the plasma membrane.

Magee T, Marshall C.

⁵⁵Nephrol Dial Transplant (2002) 17: 349-353

Statins and angiotensin II-induced vascular injury

Dechend R., Müller D., Park J. K., Fiebeler A., Haller H., Luft F. C.

⁵⁶Rinsho Byori. 1999 April;47(4):321-6.

Advances in interleukin-6 therapy

Ogata A, Nishimoto N, Yoshizaki K.

⁵⁷Cytokine Growth Factor Rev. 1997 December;8(4):241-52.

Signaling mechanisms through gp130: a model of the cytokine system.

Hirano T, Nakajima K, Hibi M

⁵⁸Int J Biochem Cell Biol. 1997 December;29(12):1401-18. IL-6-regulated transcription factors.

Akira S

⁵⁹Proc Natl Acad Sci U S A. 2001 Jul. 31;98(16):9014-9. Epub 2001 Jul. 24.

Rac1 mediates STAT3 activation by autocrine IL-6.

Faruqi T R, Gomez D, Bustelo X R, Bar-Sagi D, Reich N C.

⁶⁰Leukemia. 1993 December ;7(12):1948-54.

N-RAS gene activation in acute myeloid leukemia: association with expression of interleukin-6.

Lubbert M, Oster W, Knopf H P, McCormick F, Mertelsmann R, Herrmann F

⁶¹Journal of Leukocyte Biology, Vol 55, Issue 6 749-755

Role of the mevalonate pathway of isoprenoid synthesis in IL-8 generation by activated monocytic cells

R Terkeltaub, J Solan, M Barry, D Santoro and G M Bokoch

⁶²Atherosclerosis. 2004 February;172(2):219-28

Fluvastatin inhibits raft dependent Fcgamma receptor signalling in human monocytes.

Hillyard D Z, Jardine A G, McDonald K J, Cameron A J.

⁶³J Clin Invest. 1997 Dec. 1;100(11):2671-9.

Lovastatin and phenylacetate inhibit the induction of nitric oxide synthase and cytokines in rat primary astrocytes, microglia, and macrophages.

Pahan K, Sheikh F G, Namboodiri A M, Singh I

⁶⁴Current science, vol. 83, no. 6, 25 Sep. 2002 Mevalonate-independent pathway of isoprenoids synthesis:

A potential target in some human pathogens

Dubey V. S.

⁶⁵Skinmed. 2003 Jul-Aug.;2(4):222-8.

Assessment of etiologic agents in acne pathogenesis.

Burkhart C N, Gottwald L

⁶⁶ Journal of Bacteriology, April 2002, p. 2005-2018, Vol. 184, No. 7

Genome Sequence and Analysis of the Oral Bacterium Fusobacterium nucleatum Strain ATCC 25586

Vinayak Kapatral, Iain Anderson, Natalia Ivanova, Gary Reznik, Tamara Los, Athanasios Lykidis, Anamitra Bhattacharyya, Allen Bartman, Warren Gardner, Galina Grechkin, Lihua Zhu, Olga Vasieva, Lien Chu, Yakov Kogan, Oleg Chaga, Eugene Goltsman, Axel Bernal, Niels Larsen, Mark D'Souza, Theresa Walunas, Gordon Pusch, Robert Haselkorn, Michael Fonstein, Nikos Kyrpides, and Ross Overbeek

⁶⁷ BMJ. 1989 Mar. 25;298(6676):779-81.

Association between dental health and acute myocardial infarction.

Mattila K J, Nieminen M S, Valtonen V V, Rasi V P, Kesaniemi Y A, Syrjala S L, Jungell P S, Isoluoma M, Hietaniemi K, Jokinen M J.

⁶⁸J Cardiovasc Risk. 1999 February;6(1):7-11.

Periodontal disease and risk of fatal coronary heart and cerebrovascular diseases.

Morrison H I, Ellison L F, Taylor G W.

⁶⁹Curr Opin Microbiol. 1998 February;1(1):88-95.

Oral pathogens: from dental plaque to cardiac disease.

Meyer D H, Fives-Taylor P M.

⁷⁰Compend Contin Educ Dent. 2001 July;22(3 Spec No):34-41.

Periodontal disease, race, and vascular disease.

Desvarieux M.

⁷¹J Int Acad Periodontol. 2000 January;2(1):9-13.

Periodontal medicine: emerging concepts in pregnancy outcomes.

Champagne C M, Madianos P N, Lieff S, Murtha A P, Beck J D, Offenbacher S.

⁷²J Immunol. 1998 Jan. 1;160(1):403-9.

IL-1 and TNF antagonists inhibit the inflammatory response and bone loss in experimental periodontitis.

Assuma R, Oates T, Cochran D, Amar S, Graves D T.

⁷³Journal of Periodontology: August 1997 Vol. 68, No. 8

Treatment of Periodontal Disease in Diabetics Reduces Glycated Hemoglobin

Sara G. Grossi, Fred B. Skrepcinski, Thomas DeCaro, Don C. Robertson, Alex W. Ho, Robert G. Dunford, and Robert J. Genco

⁷⁴Lancet 2000; 355: 741-744

Prenylation inhibitors in renal disease.

Khwaja A, O'Connolly J, Hendry B M.

⁷⁵Hypertension2000; 36: 325-329

Fluvastatin inhibits matrix metalloproteinase-1 expression in human vascular endothelial cells.

Ikeda U, Shimpo M, Ohki R K et al.

⁷⁶Arch Biochem Biophys. 2000 Jan. 1;373(1):231-41.

Alendronate is a specific, nanomolar inhibitor of farnesyl diphosphate synthase.

Bergstrom J D, Bostedor R G, Masarachia P J, Reszka A A, Rodan G.

⁷⁷Curr Pharm Des. 2003;9(32):2643-58

New insights into the molecular mechanisms of action of bisphosphonates.

Rogers M J.

⁷⁸Biochem Biophys Res Commun. 2002 Jan. 18;290(2):869-73.

Identification of a bisphosphonate that inhibits isopentenyl diphosphate isomerase and farnesyl diphosphate synthase.

Thompson K, Dunford J E, Ebetino F H, Rogers M J.

⁷⁹Bone. 2003 January;32(1):15-9.

Changes in serum HDL and LDL cholesterol in patients with Paget's bone disease treated with pamidronate.

Montagnani A, Gonnelli S, Cepollaro C, Campagna M S, Franci M B, Pacini S, Gennari C.

⁸⁰Atherosclerosis. 2003 August;169(2):283-91.

Soluble intercellular adhesion molecule-1 and interleukin-6 levels reflect endothelial dysfunction in patients with primary hypercholesterolaemia treated with atorvastatin.

Nawawi H, Osman N S, Annuar R, Khalid B A, Yusoff K.

⁸¹Aust Prescriber. 2000;23(6):130-2.

Experimental and clinical pharmacology: bisphosphonates-mechanisms of action.

Martin T J G, V.

⁸²Med. 1997 Oct. 20;115(29):37-42.

[Bisphosphonate therapy in osteoporosis. Inhibition of trabecular perforation by aminobisphosphonate]. Fortschr

Wuster C, Heilmann P.

⁸³Clin Exp Rheumatol. 2002 May-June;20(3):359-64

Clodronate treatment reduces serum levels of interleukin-6 soluble receptor in Paget's disease of bone.

Rendina D, Postiglione L, Vuotto P, Numis F G, Di Domenico G, Viceconti R, Mossetti G, Nunziata V

⁸⁴Methods Find Exp Clin Pharmacol. 1999 October;21(8):519-22.

Etidronate inhibits the production of IL-6 by osteoblast-like cells.

Olmos J M, De Vega T, Perera L, Riancho J A, Amado J A, Gonzalez-Macias J.

⁸⁵Scand J Rheumatol. 1998;27(1):38-41.

Bisphosphonates inhibit IL-6 production by human osteoblast-like cells.

Giuliani N, Pedrazzoni M, Passeri G, Girasole G.

⁸⁶Endocrinology. 1996 June; 137(6):2324-33

Bisphosphonates induce osteoblasts to secrete an inhibitor of osteoclast-mediated resorption.

Vitte C, Fleisch H, Guenther H L.

⁸⁷J Clin Invest. 1999 November;104(10):1363-74

Prevention of osteocyte and osteoblast apoptosis by bisphosphonates and calcitonin.

Plotkin L I, Weinstein R S, Parfitt A M, Roberson P K, Manolagas S C, Bellido T

⁸⁸Eur J Haematol. 1998 August;61(2):128-34

Long-term oral pamidronate treatment inhibits osteoclastic bone resorption and bone turnover without affecting osteoblastic function in multiple myeloma.

Abildgaard N, Rungby J, Glerup H, Brixen K, Kassem M, Brincker H, Heickendorff L, Eriksen E F, Nielsen J L.

⁸⁹Circulation. 2002 Oct. 15;106(16):2055-60.

ARBITER: Arterial Biology for the Investigation of the Treatment Effects of Reducing Cholesterol: a randomized trial comparing the effects of atorvastatin and pravastatin on carotid intima medial thickness.

Taylor A J, Kent S M, Flaherty P J, Coyle L C, Markwood T T, Vernalis M N.

⁹⁰Am J Med. 2004 Mar. 22;116 Suppl 6A:31S-36S.

Atherosclerosis measured by B-mode ultrasonography: effect of statin therapy on disease progression.

Kastelein J J, de Groot E, Sankatsing R.

⁹¹Circulation. 74:1399-1406

Intimal plus medial thickness of the arterial wall: a direct measrement with ultrasound imaging.

Pignoli P, Tremoli E, Poli A, Oreste P, Paoletti R. 1986

⁹² Circulation. 87(Suppl 2):II56-II65.

Ultrasound B-mode imaging in observational studies of atherogenic progression.

Salonen J T, Salonen R. 1993

⁹³Diabetes Care. 17:1330-1336

Common carotid arterial wall thickness in NIDDM subjects.

Pujia A, Gnasso A, Iraace C, Colonna A, Mattioli PL. 1994

⁹⁴Lancet. 350:SI14-SI19

Non-invasive assessment of cardiovascular disease in diabetes mellitus.

Iehmann E D, Riley W A, Clarkson P, Gosling R G. 1997

⁹⁵Diabetes Care. 21:323-324

1998 Carotid arterial intimal-medial thickening and plaque formation in NIDDM [Letter].

Minamikawa J, Yamauchi M, Tanaka S, Koshiyama H.

⁹⁶ The Journal of Clinical Endocrinology & Metabolism Vol. 85, No. 8 2793-2796

Decrease in Carotid Intima-Media Thickness after 1-Year Therapy with Etidronate for Osteopenia Associated with Type 2 Diabetes

Hiroyuki Koshiyama, Yoshio Nakamura, Satsuki Tanaka and Jun Minamikawa

⁹⁷Atherosclerosis. 1987 September;67(1):41-8

The effect of ethane-1-hydroxy-1,1-diphosphonate (EHDP) on necrosis of atherosclerotic lesions.

Daoud A S, Frank A S, Jarmolych J, Fritz K E

⁹⁸Mol Genet Metab. 2004 June;82(2):180-6.

A common functional variant in the interleukin-6 gene is associated with increased body mass index in subjects with type 2 diabetes mellitus.

Stephens J W, Hurel S J, Cooper J A, Acharya J, Miller G J, Humphries S E.

⁹⁹Biochem Biophys Res Commun. 2002 Sep. 20;297(2):419-24.

Incadronate disodium inhibits advanced glycation end products-induced angiogenesis in vitro.

Okamoto T, Yamagishi S, Inagaki Y, Amano S, Takeuchi M, Kikuchi S, Ohno S, Yoshimura A.

¹⁰⁰Diabetologia. 2001 November;44(11):2032-7.

Bisphosphonates in the treatment of Charcot neuroarthropathy: a double-blind randomised controlled trial.

Jude E B, Selby P L, Burgess J, Llleystone P, Mawer E B, Page S R, Donohoe M, Foster A V, Edmonds M E, Boulton A J.

¹⁰¹Lancet. 2004 Aug. 21;364(9435):685-96.

Primary prevention of cardiovascular disease with atorvastatin in type 2 diabetes in the Collaborative Atorvastatin Diabetes Study (CARDS): multicentre randomised placebo-controlled trial.

Colhoun H M, Betteridge D J, Durrington P N, Hitman G A, Neil H A, Livingstone S J, Thomason M J, Mackness M I, Charlton-Menys V, Fuller J H; CARDS investigators.

¹⁰²Lancet 1993 Oct. 23;342(8878):1012-6

Signs and symptoms of reflex sympathetic dystrophy: prospective study of 829 patients.

Veldman P H, Reynen H M, Arntz I E, Goris R J.

¹⁰³J Rheumatol. 2003 March;30(3):474-9

Bone resorption and inflammatory inhibition efficacy of intermittent cyclical etidronate therapy in rheumatoid arthritis.

Hasegawa J, Nagashima M, Yamamoto M, Nishijima T, Katsumata S, Yoshino S

¹⁰⁴Eur J Cancer. 1997 February;33(2):304-6

Acute effects of pamidronate administration on serum levels of interleukin-6 in advanced solid tumour patients with bone metastases and their possible implications in the immunotherapy of cancer with interleukin-2.

Lissoni P, Cazzaniga M, Barni S, Perego M S, Brivio F, Fumagalli L, Tancini G.

¹⁰⁵The Biochemical Origin of Pain—2^{nd} Ed: How a new law and new drugs have led to a medical breakthrough in the treatment of Persistent pain.

Hawthorne, Calif. State-of-the-Art Technologies Publishers 2004

Omoigui S.

¹⁰⁶The Biochemical Origin of Pain—2^{nd} Ed: How a new law and new drugs have led to a medical breakthrough in the treatment of Persistent pain.

Hawthorne, Calif. State-of-the-Art Technologies Publishers 2004

Omoigui S.

¹⁰⁷The Biochemical Origin of Pain—2^{nd} Ed: How a new law and new drugs have led to a medical breakthrough in the treatment of Persistent pain.

Hawthorne, Calif. State-of-the-Art Technologies Publishers 2004

Omoigui S.

¹⁰⁸Osteoporos Int. 2003 June;14(4):273-82. Epub 2003 May 08

HMG CoA reductase inhibitors and the skeleton: a comprehensive review.

Bauer D C.

¹⁰⁹J Clin Densitom. 2002 Summer;5(2):151-8.

Effect of HMG-CoA reductase inhibitors (statins) on bone mineral density.

Funkhouser H L, Adera T, Adler R A.

¹¹⁰Arch Intern Med. 2002 Mar. 11;162(5):537-40.

Statin use, bone mineral density, and fracture risk: Geelong Osteoporosis Study.

Pasco J A, Kotowicz M A, Henry M J, Sanders K M, Nicholson G C; Geelong Osteoporosis Study.

¹¹¹J Bone Miner Res. 2003 January;18(1):88-96.

The ability of statins to inhibit bone resorption is directly related to their inhibitory effect on HMG-CoA reductase activity.

Staal A, Frith J C, French M H, Swartz J, Gungor T, Harrity T W, Tamasi J, Rogers M J, Feyen J H

¹¹²The Biochemical Origin of Pain: How a new law and new drugs have led to a medical breakthrough in the treatment of Persistent pain.

Hawthorne, Calif. State-of-the-Art Technologies Publishers 2002

Omoigui S.

¹¹³Clin Exp Rheumatol. 2004 January-February;22(1):71-8.

Effects of YM529, a novel minodronic acid, on adjuvant arthritis in rats.

Masuda-Aiba S, Shinozaki T, Takagishi K.

¹¹⁴J Pharmacol Sci. 2004 April;94(4):403-9

Simvastatin, an HMG-CoA reductase inhibitor, reduced the expression of matrix metalloproteinase-9 (Gelatinase B) in osteoblastic cells and HT1080 fibrosarcoma cells.

Thunyakitpisal P D, Chaisuparat R.

¹¹⁵J Immunol. 2003 Feb. 1;170(3):1524-30

A novel anti-inflammatory role for simvastatin in inflammatory arthritis.

Leung B P, Sattar N, Crilly A, Prach M, McCarey D W, Payne H, Madhok R, Campbell C, Gracie J A, Liew F Y, McInnes I B.

¹¹⁶Arterioscler Thromb Vasc Biol. 2001 August;21(8):1327-32

In vivo anti-inflammatory effect of statins is mediated by nonsterol mevalonate products.

Diomede L, Albani D, Sottocorno M, Donati M B, Bianchi M, Fruscella P, Salmona M.

¹¹⁷Neurology. 2003 Jul. 8;61(1):76-80

Inflammatory markers and cognition in well-functioning African-American and white elders.

Yaffe K, Lindquist K, Penninx B W, Simonsick E M, Pahor M, Kritchevsky S, Launer L, Kuller L, Rubin S, Harris T.

¹¹⁸J Nutr Health Aging. 2002;6(5):324-31

HMG-CoA reductase inhibitors (statins) in the treatment of Alzheimer's disease and why it would be ill-advise to use one that crosses the blood-brain barrier.

Sparks D L, Connor D J, Browne P J, Lopez J E, Sabbagh M N.

¹¹⁹J Mol Neurosci. 2004;23(3):225-33

Apolipoprotein E as a target for developing new therapeutics for Alzheimer's disease based on studies from protein, RNA, and regulatory region of the gene.

Lahiri D K.

¹²⁰Brain Res. 2002 Dec. 20;958(1):100-11.

Secretion of apolipoprotein E by brain glia requires protein prenylation and is suppressed by statins.

Naidu A, Xu Q, Catalano R, Cordell B.

¹²¹Eur J Neurosci. 2003 January;17(1):93-102.

Blockade of HMG-CoA reductase activity causes changes in microtubule-stabilizing protein tau via suppression of geranylgeranylpyrophosphate formation: implications for Alzheimer's disease.

Meske V, Albert F, Richter D, Schwarze J, Ohm T G.

¹²²Arch Neurol. 2002 March;59(3):378-84.

Serum lipoprotein levels, statin use, and cognitive function in older women.

Yaffe K, Barrett-Connor E, Lin F, Grady D.

¹²³Arch Neurol. 2002 February;59(2):223-7

Use of lipid-lowering agents, indication bias, and the risk of dementia in community-dwelling elderly people.

Rockwood K, Kirkland S, Hogan D B, MacKnight C, Merry H, Verreault R, Wolfson C, McDowell I.

¹²⁴International Journal of Geriatric Psychiatry Volume 17, Issue 7, Pages 601-603 Primary hyperparathyroidism in an elderly woman: surgical reversibility of profound mental state problems due to mild hypercalcaemia

Ann McDonald C., David G. Bruce D G, Smith D J

¹²⁵Am J Physiol Regul Integr Comp Physiol 286: R1013-R1023, 2004 Interleukin-6 impairs endothelium-dependent NO-cGMP-mediated relaxation and enhances contraction in systemic vessels of pregnant rats

Orshal J. M., Khalil R. A.

¹²⁶J Hum Hypertens. 2004 Sep. 9

Long-term effects of statins on arterial pressure and stiffness of hypertensives.

Ichihara A, Hayashi M, Koura Y, Tada Y, Kaneshiro Y, Saruta T.

¹²⁷Am Heart J. 2004 August; 148(2):285-92.

Association between different lipid-lowering treatment strategies and blood pressure control in the Brisighella Heart Study.

Borghi C, Dormi A, Veronesi M, Sangiorgi Z, Gaddi A; Brisighella Heart Study Working Party.

¹²⁸Kiecolt-Glaser J K, Preacher K J, MacCallum R C, Atkinson C, Malarkey W B, Glaser R. Chronic stress and age-related increases in the proinflammatory cytokine IL-6.

Proc Natl Acad Sci U S A. 2003 Jul. 22;100(15):9090-5. Epub 2003 Jul. 02.

I claim:

1. A method of prevention and treatment of vascular and age-related disorders by synergistic inhibition or reduction of Interleukin-6 inflammation through regulation of cholesterol metabolism and isoprenoid depletion, in a human or other animal subject. Said method comprises administering, to said subject, any one of the following combinations of components that are regulators of cholesterol metabolism and interleukin-6 mediated inflammation:
I. AandB
II. A, B, and C
I. A and C
IV. B and C
Wherein
A is a HMG-CoA reductase inhibitor selected from the group including of lovastatin, simvastatin, pravastatin, fluvastatin, atorvastatin, rivastatin, red yeast rice, red yeast grain, red yeast powder and other statins or a pharmaceutically acceptable salt thereof.
B. is a bisphosphonate selected from the group including of Pamidronate, Etidronate, Clodronate, Alendronate, phosphonic acid derivatives, an ester thereof, a pharmaceutically acceptable salt thereof, a hydrate thereof
C. is a cholesterol lowering agent or technique selected from the group including of (i) low cholesterol or low fat diet (ii) sequestrants (cholestyramine, colestipol and dialkylaminoalkyl derivatives of a cross-linked dextran), (iii) nicotinyl alcohol, nicotinic acid or a salt thereof, (iv) PPAR.alpha. agonists such as fenofibric acid derivatives (gemfibrozil, clofibrate, fenofibrate and benzafibrate), (v) inhibitors of cholesterol absorption for example beta-sitosterol and (acyl CoA:cholesterol acyltransferase) inhibitors for example melinamide and (vi) probucol, an ester thereof, a pharmaceutically acceptable salt thereof, a hydrate thereof.
said components being administered simultaneously or separately, in amounts which have the effect of ameliorating the vascular and age-related disorders.

2. The method of claim 1, wherein;
a) said vascular and age-related disorder is atherosclerosis.
b) a therapeutically effective amount of said combinations of regulators of cholesterol metabolism and interleukin-6 mediated inflammation is administered subcutaneously, intramuscularly, intravenously, orally or rectally

3. The method of claim 1, wherein;
a) said vascular and age-related disorder is peripheral vascular disease.
b) a therapeutically effective amount of said combinations of regulators of cholesterol metabolism and interleukin-6 mediated inflammation is administered subcutaneously, intramuscularly, intravenously, orally or rectally

4. The method of claim 1, wherein;
a) said vascular and age-related disorder is coronary artery disease.
b) a therapeutically effective amount of said combinations of regulators of cholesterol metabolism and interleukin-6 mediated inflammation is administered subcutaneously, intramuscularly, intravenously, orally or rectally

5. The method of claim 1, wherein;
a) said vascular and age-related disorder is osteoporosis.
b) a therapeutically effective amount of said combinations of regulators of cholesterol metabolism and interleukin-6 mediated inflammation is administered subcutaneously, intramuscularly, intravenously, orally or rectally.

6. The method of claim 1, wherein;
a) said vascular and age-related disorder is arthritis.
b) a therapeutically effective amount of said combinations of regulators of cholesterol metabolism and interleukin-6 mediated inflammation is administered subcutaneously, intramuscularly, intravenously, orally or rectally.

7. The method of claim 1, wherein;
a) said vascular and age-related disorder is Type 1 diabetes, Type 2 diabetes, inadequate glucose tolerance or insulin resistance.
b) a therapeutically effective amount of said combinations of regulators of cholesterol metabolism and interleukin-6 mediated inflammation is administered subcutaneously, intramuscularly, intravenously, orally or rectally.

8. The method of claim 1, wherein;
a) said vascular and age-related disorder is obesity
b) a therapeutically effective amount of said combinations of regulators of cholesterol metabolism and interleukin-6 mediated inflammation is administered subcutaneously, intramuscularly, intravenously, orally or rectally.

9. The method of claim 1, wherein;
a) said vascular and age-related disorder is hypertension
b) a therapeutically effective amount of said combinations of regulators of cholesterol metabolism and interleukin-6 mediated inflammation is administered subcutaneously, intramuscularly, intravenously, orally or rectally.

10. The method of claim 1, wherein;
a) said vascular and age-related disorder is dementia
b) a therapeutically effective amount of said combinations of regulators of cholesterol metabolism and interleukin-6 mediated inflammation is administered subcutaneously, intramuscularly, intravenously, orally or rectally.

11. The method of claim 1, wherein;
a) said vascular and age-related disorder is Alzheimer's disease
b) a therapeutically effective amount of said combinations of regulators of cholesterol metabolism and interleukin-6 mediated inflammation is administered subcutaneously, intramuscularly, intravenously, orally or rectally.

12. The method of claim 1, wherein;
a) said vascular and age-related disorder is Aging
b) a therapeutically effective amount of said combinations of regulators of cholesterol metabolism and interleukin-6 mediated inflammation is administered subcutaneously, intramuscularly, intravenously, orally or rectally.

13. The method of claim 1, wherein;
a) said vascular and age-related disorder is Periodontal disease or other chronic low grade infection such as Chlamydia pneumoniae
b) a therapeutically effective amount of said combinations of regulators of cholesterol metabolism and interleukin-6 mediated inflammation is administered subcutaneously, intramuscularly, intravenously, orally or rectally.

14. A method of prevention and treatment of vascular and age-related disorders by inhibition or reduction of Interleukin-6 inflammation through regulation of cholesterol metabolism and isoprenoid depletion, in a human or other animal subject. Said method comprises administering, to said subject, in amounts which have the effect of ameliorating the vascular and age-related disorders, a HMG-CoA reductase inhibitor selected from the group including of lovastatin, simvastatin, pravastatin, fluvastatin, atorvastatin, rivastatin, red yeast rice, red yeast grain, red yeast powder and other statins or a pharmaceutically acceptable salt thereof.

15. The method of claim 14, wherein;
a) said vascular and age-related disorder is peripheral vascular disease.
b) a therapeutically effective amount of said regulator of cholesterol metabolism and interleukin-6 mediated inflammation is administered subcutaneously, intramuscularly, intravenously, orally or rectally.

16. The method of claim 14, wherein;
a) said vascular and age-related disorder is coronary artery disease.
b) a therapeutically effective amount of said regulator of cholesterol metabolism and interleukin-6 mediated inflammation is administered subcutaneously, intramuscularly, intravenously, orally or rectally.

17. The method of claim 14, wherein;
a) said vascular and age-related disorder is arthritis.
b) a therapeutically effective amount of said regulator of cholesterol metabolism and interleukin-6 mediated inflammation is administered subcutaneously, intramuscularly, intravenously, orally or rectally.

18. The method of claim 14, wherein;
a) said vascular and age-related disorder is Type 1 diabetes, Type 2 diabetes, inadequate glucose tolerance or insulin resistance.
b) a therapeutically effective amount of said regulator of cholesterol metabolism and interleukin-6 mediated inflammation is administered subcutaneously, intramuscularly, intravenously, orally or rectally.

19. The method of claim 14, wherein;
a) said vascular and age-related disorder is obesity
b) a therapeutically effective amount of said regulator of cholesterol metabolism and interleukin-6 mediated inflammation is administered subcutaneously, intramuscularly, intravenously, orally or rectally.

20. The method of claim 14, wherein;
a) said vascular and age-related disorder is hypertension
b) a therapeutically effective amount of said regulator of cholesterol metabolism and interleukin-6 mediated inflammation is administered subcutaneously, intramuscularly, intravenously, orally or rectally.

21. The method of claim 14, wherein;
a) said vascular and age-related disorder is dementia
b) a therapeutically effective amount of said regulator of cholesterol metabolism and interleukin-6 mediated inflammation is administered subcutaneously, intramuscularly, intravenously, orally or rectally.

22. The method of claim 14, wherein;
a) said vascular and age-related disorder is Alzheimer's disease
b) a therapeutically effective amount of said regulator of cholesterol metabolism and interleukin-6 mediated inflammation is administered subcutaneously, intramuscularly, intravenously, orally or rectally.

23. The method of claim 14, wherein;
a) said vascular and age-related disorder is Aging
b) a therapeutically effective amount of said regulator of cholesterol metabolism and interleukin-6 mediated inflammation is administered subcutaneously, intramuscularly, intravenously, orally or rectally.

24. The method of claim 14, wherein;
a) said vascular and age-related disorder is Periodontal disease or other chronic low grade infection such as Chlamydia pneumoniae
b) a therapeutically effective amount of said combinations of regulators of cholesterol metabolism and interleukin-6 mediated inflammation is administered subcutaneously, intramuscularly, intravenously, orally or rectally.

25. A method of prevention and treatment of vascular and age-related disorders by inhibition or reduction of Interleukin-6 inflammation through regulation of cholesterol metabolism and isoprenoid depletion, in a human or other animal subject. Said method comprises administering, to said subject, in amounts which have the effect of ameliorating the vascular and age-related disorders, a bisphosphonate selected from the group including of Pamidronate, Etidronate, Clodronate, Alendronate, phosphonic acid derivatives, an ester thereof, a pharmaceutically acceptable salt thereof, a hydrate thereof.

26. The method of claim 25, wherein;
a) said vascular and age-related disorder is peripheral vascular disease.
b) a therapeutically effective amount of said regulator of cholesterol metabolism and interleukin-6 mediated inflammation is administered subcutaneously, intramuscularly, intravenously, orally or rectally.

27. The method of claim 25, wherein;
a) said vascular and age-related disorder is coronary artery disease.
b) a therapeutically effective amount of said regulator of cholesterol metabolism and interleukin-6 mediated inflammation is administered subcutaneously, intramuscularly, intravenously, orally or rectally.

28. The method of claim 25, wherein;
a) said vascular and age-related disorder is arthritis.
b) a therapeutically effective amount of said regulator of cholesterol metabolism and interleukin-6 mediated inflammation is administered subcutaneously, intramuscularly, intravenously, orally or rectally.

29. The method of claim 25, wherein;
a) said vascular and age-related disorder is Type 1 diabetes, Type 2 diabetes, inadequate glucose tolerance or insulin resistance.
b) a therapeutically effective amount of said regulator of cholesterol metabolism and interleukin-6 mediated inflammation is administered subcutaneously, intramuscularly, intravenously, orally or rectally.

30. The method of claim 25, wherein;
a) said vascular and age-related disorder is obesity
b) a therapeutically effective amount of said regulator of cholesterol metabolism and interleukin-6 mediated inflammation is administered subcutaneously, intramuscularly, intravenously, orally or rectally.

31. The method of claim 25, wherein;
a) said vascular and age-related disorder is hypertension
b) a therapeutically effective amount of said regulator of cholesterol metabolism and interleukin-6 mediated inflammation is administered subcutaneously, intramuscularly, intravenously, orally or rectally.

32. The method of claim 25, wherein;
a) said vascular and age-related disorder is dementia
b) a therapeutically effective amount of said regulator of cholesterol metabolism and interleukin-6 mediated inflammation is administered subcutaneously, intramuscularly, intravenously, orally or rectally.

33. The method of claim 25, wherein;
a) said vascular and age-related disorder is Alzheimer's disease
b) a therapeutically effective amount of said regulator of cholesterol metabolism and interleukin-6 mediated inflammation is administered subcutaneously, intramuscularly, intravenously, orally or rectally.

34. The method of claim 25, wherein;
a) said vascular and age-related disorder is Aging
b) a therapeutically effective amount of said regulator of cholesterol metabolism and interleukin-6 mediated inflammation is administered subcutaneously, intramuscularly, intravenously, orally or rectally.

35. The method of claim 25, wherein;
a) said vascular and age-related disorder is Periodontal disease or other chronic low grade infection such as Chlamydia pneumoniae
b) a therapeutically effective amount of said combinations of regulators of cholesterol metabolism and interleukin-6 mediated inflammation is administered subcutaneously, intramuscularly, intravenously, orally or rectally.

36. A method of prevention and treatment of vascular and age-related disorders by inhibition or reduction of Interleukin-6 inflammation through regulation of cholesterol metabolism and isoprenoid depletion, in a human or other animal subject. Said method comprises administering, to said subject, in amounts which have the effect of ameliorating the vascular and age-related disorders, a cholesterol lowering agent or technique selected from the group including of (i) low cholesterol or low fat diet (ii) sequestrants (cholestyramine, colestipol and dialkylaminoalkyl derivatives of a cross-linked dextran), (iii) nicotinyl alcohol, nicotinic acid or a salt thereof, (iv) PPAR.alpha. agonists such as fenofibric acid derivatives (gemfibrozil, clofibrate, fenofibrate and benzafibrate), (v) inhibitors of cholesterol absorption for example beta-sitosterol and (acyl CoA:cholesterol acyltransferase) inhibitors for example melinamide and (vi) probucol, an ester thereof, a pharmaceutically acceptable salt thereof, a hydrate thereof.

37. The method of claim 36, wherein;
a) said vascular and age-related disorder is peripheral vascular disease.
b) a therapeutically effective amount of said regulator of cholesterol metabolism and interleukin-6 mediated inflammation is administered subcutaneously, intramuscularly, intravenously, orally or rectally.

38. The method of claim 36, wherein;
a) said vascular and age-related disorder is coronary artery disease.
b) a therapeutically effective amount of said regulator of cholesterol metabolism and interleukin-6 mediated inflammation is administered subcutaneously, intramuscularly, intravenously, orally or rectally.

39. The method of claim 36, wherein;
a) said vascular and age-related disorder is arthritis.
b) a therapeutically effective amount of said regulator of cholesterol metabolism and interleukin-6 mediated inflammation is administered subcutaneously, intramuscularly, intravenously, orally or rectally.

40. The method of claim 36, wherein;
a) said vascular and age-related disorder is Type 1 diabetes, Type 2 diabetes, inadequate glucose tolerance or insulin resistance.
b) a therapeutically effective amount of said regulator of cholesterol metabolism and interleukin-6 mediated inflammation is administered subcutaneously, intramuscularly, intravenously, orally or rectally.

41. The method of claim 36, wherein;
a) said vascular and age-related disorder is obesity
b) a therapeutically effective amount of said regulator of cholesterol metabolism and interleukin-6 mediated inflammation is administered subcutaneously, intramuscularly, intravenously, orally or rectally.

42. The method of claim 36, wherein;
a) said vascular and age-related disorder is hypertension
b) a therapeutically effective amount of said regulator of cholesterol metabolism and interleukin-6 mediated inflammation is administered subcutaneously, intramuscularly, intravenously, orally or rectally.

43. The method of claim 36, wherein;
a) said vascular and age-related disorder is dementia
b) a therapeutically effective amount of said regulator of cholesterol metabolism and interleukin-6 mediated inflammation is administered subcutaneously, intramuscularly, intravenously, orally or rectally.

44. The method of claim 36, wherein;
a) said vascular and age-related disorder is Alzheimer's disease
b) a therapeutically effective amount of said regulator of cholesterol metabolism and interleukin-6 mediated inflammation is administered subcutaneously, intramuscularly, intravenously, orally or rectally.

45. The method of claim 36, wherein;
a) said vascular and age-related disorder is Aging
b) a therapeutically effective amount of said regulator of cholesterol metabolism and interleukin-6 mediated inflammation is administered subcutaneously, intramuscularly, intravenously, orally or rectally.

46. The method of claim 36, wherein;
a) said vascular and age-related disorder is Periodontal disease or other chronic low grade infection such as Chlamydia pneumoniae
b) a therapeutically effective amount of said combinations of regulators of cholesterol metabolism and interleukin-6 mediated inflammation is administered subcutaneously, intramuscularly, intravenously, orally or rectally.
